SEQUENCE LISTING (6 sequences)

SEQ ID NO: 1 - RNA, 20 nt, Artificial Sequence
  IRAK4 forward primer
gctgcctcaatgttggacta

SEQ ID NO: 2 - RNA, 20 nt, Artificial Sequence
  IRAK4 reverse primer
tctggacttgaggagtcagg

SEQ ID NO: 3 - RNA, 21 nt, Artificial Sequence
  CXCL2 forward primer
cgcccatggttaagaaaatca

SEQ ID NO: 4 - RNA, 22 nt, Artificial Sequence
  CXCL2 reverse primer
ccttctggtcagttggatttgc

SEQ ID NO: 5 - RNA, 20 nt, Artificial Sequence
  CXCL8 forward primer
cttggcagccttcctgattt

SEQ ID NO: 6 - RNA, 23 nt, Artificial Sequence
  CXCL8 reverse primer
ttctttagcactccttggcaaaa

(19) United States
(12) Patent Application Publication
(10) Pub. No.: US 2021/0292843 A1
(43) Pub. Date: Sep. 23, 2021

(54) TREATMENT OF DISEASES ASSOCIATED WITH ACTIVATED IRAK

(71) Applicant: CHILDREN'S HOSPITAL MEDICAL CENTER, Cincinnati, OH (US)

(72) Inventors: Molly SMITH, Cincinnati, OH (US); Daniel STARCZYNOWSKI, Cincinnati, OH (US); Kakajan KOMUROV, West Chester, OH (US)

(21) Appl. No.: 16/339,692

(22) PCT Filed: Oct. 30, 2017
(86) PCT No.: PCT/US2017/059091

§ 371 Date: Apr. 4, 2019

Related U.S. Application Data
(60) Provisional application No. 62/414,058, filed on Oct. 28, 2016.
(60) Provisional application No. 62/429,289, filed on Dec. 2, 2016.

(51) Int. Cl. C12Q 1/6886; A61K 45/06

(52) U.S. Cl. CPC C12Q 1/6886; C12Q 2600/156; C12Q 2600/158; A61K 45/06

(57) ABSTRACT

Methods and compositions disclosed herein generally relate to compositions and methods for the treatment of cancers, including disorders such as myelodysplastic syndrome (MDS) and acute myeloid leukemia (AML). In particular, the invention relates to determining an individual in need of treatment who can be treated with an IRAK1/4 inhibitor.

### CROSS REFERENCE TO RELATED APPLICATION

The present application claims the benefit of priority under 35 U.S.C. § 119(e) to U.S. Provisional Application No. 62/414,058, OVER-EXPRESSION OF U2AF1 AS A GENETIC PREDICTOR OF ACTIVATED IRAK, filed on Oct. 28, 2016, and U.S. Provisional Application No. 62/429,289, OVER-EXPRESSION OF U2AF1 AS A GENETIC PREDICTOR OF ACTIVATED IRAK, filed on Dec. 2, 2016, which are currently co-pending herewith and which are incorporated by reference in their entirety.

### STATEMENT REGARDING FEDERALLY-SPONSORED RESEARCH

This invention was made with government support under Grant No. HL132420 awarded by the National Institutes of Health (NIH). The government has certain rights in the invention.

### SEQUENCE LISTING

A Sequence Listing comprising SEQ ID NOS:1-6, has been provided in computer readable form (.txt) as part of this application, and is incorporated by reference herein in its entirety as part of this application.

### FIELD OF THE INVENTION

The invention disclosed herein generally relates to treatment of cancer and determining an appropriate treatment for a subject having cancer.

### BACKGROUND

Myelodysplastic syndromes (MDS) are malignant, potentially fatal blood diseases that arise from a defective hematopoietic stem/progenitor cell and often progress to chemotherapy-resistant secondary acute myeloid leukemia (sAML). MDS are heterogeneous diseases with few treatment options. One of the key challenges facing MDS treatment is the lack of effective medicines capable of providing a durable response.

MDS are hematologic malignancies defined by blood cytopenias resulting from ineffective hematopoiesis; MDS further confers a predisposition to acute myeloid leukemia (AML) (Corey et al., 2007; Nimer, 2008). Senior citizens are more susceptible to MDS, and the incidence of MDS has escalated in recent years as a result of longer life expectancies (Sekeres, 2010b). A majority of patients having MDS die of marrow failure, immune dysfunction, and/or transformation to overt leukemia.

Current treatment options for MDS are limited but include allogeneic HSC transplantation, demethylating agents, and immunomodulatory therapies (Ebert, 2010). While hemopoeitic stem cell (HSC) transplantation can be used as a curative treatment for MDS, this option is unavailable to many older patients, who instead receive supportive care and transfusions to ameliorate disease complications. Unfortunately, MDS clones can persist in the marrow even after HSC transplantation, and the disease invariably advances (Tehranchi et al., 2010). For advanced disease or high-risk MDS, patients may also receive immunosuppressive therapy, epigenetic modifying drugs, and/or chemotherapy (Greenberg, 2010). Despite recent progress, most MDS patients exhibit treatment-related toxicities or relapse (Sekeres, 2010a). Overall, the efficacy of these treatments is variable, and generally life expectancies are only slightly improved as compared to supportive care. The complexity and heterogeneity of MDS, and the lack of human xenograft models are obstacles which are challenging for identifying and evaluating novel molecular targets for this disease.

Approximately 30% of MDS patients also develop aggressive AML due to acquisition of additional mutations in the defective hematopoietic stem/progenitor cell (HSPC) (Greenberg et al., 1997). AML is a cancer of the myeloid line of blood cells, characterized by the rapid growth of abnormal white blood cells that accumulate in the bone marrow and interfere with the production of normal blood cells. AML is the most common acute leukemia affecting adults, and its incidence increases with age. Although AML is a relatively rare disease, accounting for approximately 1.2% of cancer deaths in the United States, its incidence is expected to increase as the population ages. Several risk factors and chromosomal abnormalities have been identified, but the specific cause is not clear. As an acute leukemia, AML progresses rapidly and is typically fatal within weeks or months if left untreated. The prognosis for AML that arises from MDS has a worse as compared to other types of AML.

It is therefore necessary to develop treatments and methods of effectively treating MDS and AML. Additionally, in doing so, it will be important to determine whether a patient is likely to be responsive to a particular treatment or method of treatment.

### SUMMARY OF THE INVENTION

Methods and compositions described herein are provided by way of example and should not in any way limit the scope of the invention.

Embodiments of the invention encompass methods of treating a subject having a disease or disorder, the methods including: identifying a subject having a U2AF1 mutation and/or enhanced IRAK4-Long expression and/or activity relative to IRAK4-Short, as compared to a normal control; and administering to the subject a composition including an IRAK inhibitor.

Embodiments of the invention also encompass methods of assigning a subject having a disease or disorder to a specific treatment cohort, the methods including: determining, using a test sample from a subject having or suspected of having a disease or disorder, a presence or absence of a U2AF1 mutation and/or enhanced IRAK4-Long expression and/or activity relative to IRAK4-Short, as compared to a normal control; assigning, where the U2AF1 mutation and/or enhanced IRAK4-Long is present, the sample to a first treatment cohort wherein the first treatment cohort is treatable by administration of an IRAK inhibitor; and providing the cohort assignment information to a treatment facility. In some embodiments, the methods further include assigning, where the U2AF1 mutation and/or enhanced IRAK4-Long is absent, the sample to a second treatment cohort, wherein the second treatment cohort is not treatable, or less effectively treatable by administration of the IRAK inhibitor. In some embodiments, the methods further include administering the IRAK inhibitor to the subject if the subject is in the first treatment cohort. In some embodiments, the methods further include administration of an IRAK1/4 inhibitor to the first treatment cohort. In some embodiments, the subject in the first treatment cohort or the second treatment cohort can be enrolled in a clinical trial.

Embodiments of the invention also encompass methods for improving a clinical trial for treating a disease or disorder, the methods including: determining, using a test sample from one or more subjects having or suspected of having a disease or disorder, a presence or absence of a U2AF1 mutation and/or enhanced IRAK4-Long expression and/or activity relative to IRAK4-Short, as compared to a normal control; assigning, where the U2AF1 mutation and/or enhanced IRAK4-Long is present, the sample to a first treatment cohort, and assigning, where the U2AF1 mutation and/or enhanced IRAK4-Long is absent, the sample to a second treatment cohort; and administering an IRAK inhibitor to a subject in the first treatment cohort. In some embodiments of the methods, the first treatment cohort is treatable by administration of an IRAK inhibitor, and the second treatment cohort is not treatable, or less effectively treatable by administration of the IRAK inhibitor. In some embodiments, an IRAK1/4 inhibitor is administered to the first treatment cohort.

In some embodiments, the U2AF1 mutation can be a U2AF1-S34F mutation.

In some embodiments, the IRAK inhibitor inhibits IRAK4-Long activity. In some embodiments, the IRAK inhibitor can be an IRAK1/4 inhibitor. In some embodiments, the IRAK1/4 inhibitor can be an inhibitor of IRAK4-Long activity.

In some embodiments, the disease or disorder is associated with increased IRAK4-Long expression relative to IRAK4-Short, and increased NF-kB.

In some embodiments, the disease or disorder includes myelodysplastic syndrome (MDS) or acute myeloid leukemia (AML). In some embodiments, the subject has MDS including Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del(5q)-syndrome, chronic myelomonocytic leukemia (CMML), juvenile myelomonocytic leukemia (JMML), refractory cytopenia of childhood, or a combination thereof. In some embodiments, the subject has AML including AML with recurrent genetic abnormalities (AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3), AML (megakaryoblastic) with a translocation between chromosomes 1 and 22, AML with myelodysplasia-related changes, AML related to previous chemotherapy or radiation (alkylating agent-related AML, topoisomerase II inhibitor-related AML), AML not otherwise categorized (AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis), myeloid sarcoma (also known as granulocytic sarcoma, chloroma or extramedullary myeloblastoma), undifferentiated and biphenotypic acute leukemias (also known as mixed phenotype acute leukemias), or a combination thereof. In some embodiments, administration of an IRAK inhibitor to a subject having the U2AF1 mutation decreases the incidence of one or more symptoms associated with MDS or AML or decreases one or more markers of viability of MDS or AML cells. In some embodiments, the one or more symptoms associated with MDS or AML include decreasing marrow failure, immune dysfunction, transformation to overt leukemia, or a combination thereof in the subject, or wherein the marker of viability of MDS or AML cells includes survival over time, proliferation, growth, migration, formation of colonies, chromatic assembly, DNA binding, RNA metabolism, cell migration, cell adhesion, inflammation, or a combination thereof.

In some embodiments, the disease or disorder can be a type of cancer including breast cancer, cervical cancer, colorectal cancer, endometrial cancer, glioma, head and neck cancer, liver cancer, melanoma, ovarian cancer, pancreatic cancer, prostate cancer, renal cancer, stomach cancer, testicular cancer, thyroid cancer, or urothelial cancer.

Some embodiments of the methods further include administration of an agent selected from an apoptotic agent, an immune modulating agent, an epigenetic modifying agent, or a combination thereof.

In some embodiments, only a fraction of AML or MDS subjects have a U2AF1 mutation that enhances IRAK4-Long expression relative to IRAK4-Short expression. In some embodiments, the fraction of AML or MDS subjects having a U2AF1 mutation that enhances IRAK4-Long expression relative to IRAK4-Short expression is selected from the group consisting of less than 50%, less than 25%, less than 20%, less than 15%, less than 14%, less than 13%, less than 12%, less than 11%, less than 10%, less than 9%, less than 8%, less than 7%, less than 5%, less than 4%, less than 3%, less than 2%, or less than 1%.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 shows the process whereby alternative splicing generates gene expression diversity.

FIG. 2 shows the features of interleukin receptor associated kinase 4 (IRAK4). (A) IRAK4 and IRAK1 are downstream of TLR signaling. (B) IRAK4 has three functional domains of IRAK 4. (C) IRAK4 variants encode two protein isoforms.

FIG. 3 shows that a subset of genes exhibits changes in isoform usage. (A) Two distinct isoforms have differential, and substantial, expression across all samples. (B) A subset of genes exhibits changes in isoform usage in various cancers; dots represent genes plotted by cumulative variance vs average isoform variance.

FIG. 4 shows that a subset of genes exhibits changes in isoform usage in AML and are involved in innate immune signaling. (A) Dots represent genes plotted by cumulative variance vs isoform variance. (black=genes that one main isoform contributes to the overall expression; grey=genes that multiple isoforms contribute to the cumulative expression, indicating that these genes undergo changes in isoform usage.) (B) Genes that undergo the greatest changes in isoform switching (n=402) were analyzed for enriched pathways. (C) Genes involved in enriched pathways. (D) IRAK4 variants within the coding region encode two protein isoforms, Long (IRAK4-L) and Short (IRAK4-S).

FIG. 5 shows that knockdown of IRAK4 inhibits leukemic progenitor function.

FIG. 6 shows that IRAK4 mRNA variants encode two protein isoforms in AML. (A) MDS, AML and normal bone marrow (NBM) samples were PCR amplified for IRAK4-L or IRAK4-S based on the presence or absence of exon 4 and distinguished by size using primers in exon 3 and 5. (B) The IRAK4 exon 4 cassette was confirmed by sequencing. (C and D) NBM and MDS/AML cell lines were immunoblotted for expression of long and short IRAK4 isoforms.

FIG. 7 shows that inclusion of IRAK4 exon 4 is associated with AML. (A) Ratio of long IRAK4 (IRAK4-L) to short IRAK4 (IRAK4-S) isoform expression from the Cancer Genome Atlas. (B) Kaplan-Meier curve of AML and MDS patients stratified based on IRAK4 isoform expression (as measured by presence of exon 4 in AML or by expression of the long isoform in MDS). (C) IRAK4 mRNA variants are differentially expressed in various human cancers, including AML.

FIG. 8 shows that IRAK4-L protein exhibits maximal activation of innate immune and NF-kB signaling. (A) Several gene networks were found to be enriched in IRAK4-L expressing AML patients. (B) Pathway analysis shows enriched pathways in AML patients with high expression of IRAK4-L relative to IRAK4-S(black), and high expression of IRAK4-S relative to IRAK4-L (white). (C) 293T cells transfected with FLAG-IRAK4-L or FLAG-IRAK4-S were immunoblotted using the indicated antibodies. (D) NF-kB reporter activity (kB-Luciferase) measured in 293T cells transfected with FLAG-IRAK4-L or FLAG-IRAK4-S. (E) and (F) IRAK4-L differentially regulates NF-kB and MAPK as compared to IRAK4-S.

FIG. 9 shows that inclusion of IRAK4 exon 4 is associated with U2AF1 mutations in AML, and MDS. (A) Genetic alterations correlated with IRAK4 exon 4 retention in AML. (B) Experimental design for determining U2AF1's control of splicing.

FIG. 10 shows that the U2AF1-S34F mutation correlates with increased IRAK4-L. (A) An increased amount of IRAK4-S is present with the wild-type U2AF1 gene, whereas a significantly increased amount of IRAK4-L is present with the U2AF1-S34F mutation. (B) The mutant U2AF1 confers increased exon retention.

FIG. 11 shows that U2AF1-S34F directly regulates inclusion of IRAK4 exon 4 and expression of IRAK4-L protein. (A) Experimental design to measure RNA splicing changes in human CD34+ cells transduced with WT or mutant U2AF1. RNA-sequencing junction reads for IRAK4 exon 3-4 are shown. (B) 293T cells expressing an IRAK4 exon 4 minigene cassette were transfected with WT or mutant U2AF1 and PCR amplified for exon retention (top band) and exon exclusion (bottom band). Bar graph represents intensity of top PCR band over total intensity relative to vector. (C and D) K562 cells express FLAG-U2AF1 or FLAG-U2AF1-S34F under the control of a doxycycline (DOX)-inducible promoter. (C) IRAK4 exon 4 usage was determined by RT-PCR. (D) IRAK4 and U2AF1 protein expression was determined by immunoblotting.

FIG. 12 shows that U2AF1-mutant AML cells exhibit increased innate immune pathway activation. (A) Overview of innate immune signaling. (B) K562 cells expressing wild-type or mutant U2AF1 were immunoblotted using the indicated antibodies. (C) K562 cells expressing wild-type or mutant U2AF1 were stimulated with IL-1β and examined for NF-kB target genes by qRT-PCR.

FIG. 13 shows that U2AF1-mutant AML cells are sensitive to IRAK1/4-inhibitors. (A) K562-U2AF1-S34F cells were treated with an IRAK1/4 inhibitor for 1 hour (+) and 2 hours (++). (B) K562 cells expressing wild-type or mutant U2AF1 were treated with DMSO or IRAK1/4 inhibitor over 7 days. (C) K562 cells expressing wild-type or mutant U2AF1 were evaluated for leukemic progenitor function in methylcellulose after treatment with DMSO or IRAK1/4 inhibitor for 48 hours.

### DETAILED DESCRIPTION OF THE INVENTION

All references cited herein are incorporated by reference in their entirety. Also incorporated herein by reference in their entirety include: U.S. patent application Ser. No. 15/288,402, COMBINATION THERAPY FOR MDS, filed on Oct. 7, 2016; U.S. application Ser. No. 14/842,049, COMBINATION THERAPY FOR MDS, filed Sep. 1, 2015; U.S. application Ser. No. 14/284,521, COMBINATION THERAPY FOR MDS, filed May 22, 2014; U.S. Provisional Application No. 61/826,211, filed May 22, 2013; International Patent Application No. PCT/US2016/058864, METHODS AND COMPOSITIONS FOR THE TREATMENT OF HEAD AND NECK CANCER, filed on Oct. 26, 2016; U.S. Provisional Application No. 62/248,050, METHODS AND COMPOSITIONS FOR THE TREATMENT OF HEAD AND NECK CANCER, filed on Oct. 29, 2015; International Patent Application No. WO2017______, COMPOUNDS, COMPOSITIONS, METHODS FOR TREATING DISEASES, AND METHODS FOR PREPARING COMPOUNDS, filed on ______, 2017; and U.S. Provisional Application No. 62/248,050, COMPOUNDS, COMPOSITIONS, METHODS FOR TREATING DISEASES, AND METHODS FOR PREPARING COMPOUNDS, filed on Aug. 17, 2016.

Unless otherwise noted, terms are to be understood according to conventional usage by those of ordinary skill in the relevant art.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or microdissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing,” “analyzing,” and “assaying” can be used interchangeably and can include quantitative and/or qualitative determinations.

As used herein, the term “monitoring” with reference to a type of cancer refers to a method or process of determining the severity or degree of the type of cancer or stratifying the type of cancer based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to survival/mortality over a given time horizon. For example, “outcome” can refer to survival/mortality over 1 month, 3 months, 6 months, 1 year, 5 years, or 10 years or longer. In some embodiments, an increased risk for a poor outcome indicates that a therapy has had a poor efficacy, and a reduced risk for a poor outcome indicates that a therapy has had a good efficacy.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a pediatric patient is a patient under 18 years of age, while an adult patient is 18 or older.

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the invention, a reference pattern of expression levels is, for example, an average pattern of expression levels observed in a group of healthy or diseased individuals, serving as a reference group.

As used herein, an mRNA “isoform” is an alternative transcript for a specific mRNA or gene. This term includes pre-mRNA, immature mRNA, mature mRNA, cleaved or otherwise truncated, shortened, or aberrant mRNA, modified mRNA (e.g. containing any residue modifications, capping variants, polyadenylation variants, etc.), and the like.

“Antibody” or “antibody peptide(s)” refer to an intact antibody, or a binding fragment thereof that competes with the intact antibody for specific binding; this definition also encompasses monoclonal and polyclonal antibodies. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody, for example, substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

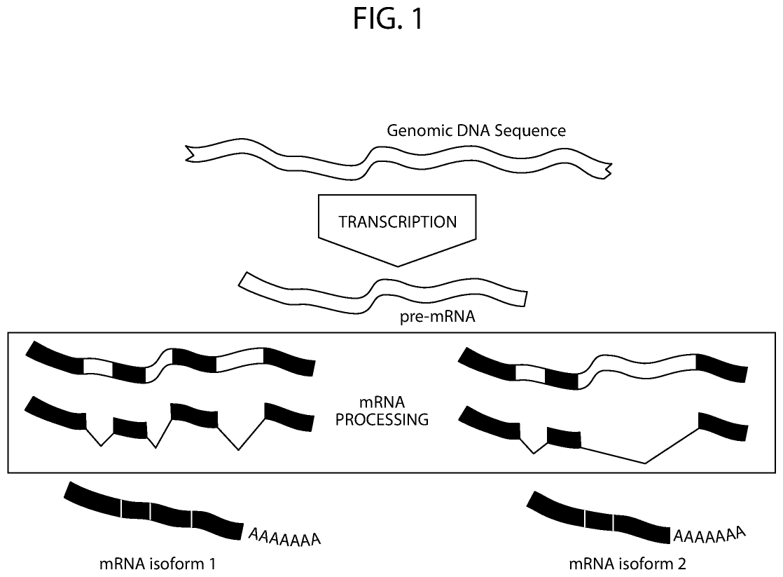

After genomic DNA is transcribed into pre-mRNA, mRNA processing takes place. During mRNA processing, alternative mRNA splicing can occur, to generate multiple mRNA isoforms from a single gene, resulting in gene expression diversity, as shown in FIG. 1.

Many genes are susceptible to changes in isoform usage, often including genes involved in innate immune signaling. Mutations in spliceosome genes and aberrant RNA splicing are common features of human cancer, particularly myelodysplastic syndromes (MDS) and acute myeloid leukemia (AML). For example, recurring mutations of spliceosome genes in MDS and AML most frequently involve the U2AF1, SF3B1, and SRSF2 genes. However, it has heretofore been unclear as to which mis-spliced genes contribute a malignant state.

MDS are genetically defined by somatic mutations and chromosomal abnormalities not only affecting epigenetic plasticity, ribosome function, spliceosome machinery, or activation of oncogenes but also immune dysfunction. Human miR-146a resides on chromosome 5q33.3, and its deletion occurs in 80% of all del(5q) MDS and AML (Gondek et al., 2008). Low expression of miR-146a, also occurs in >25% of all MDS and in >10% of AML patients (Sokol et al., 2011; Starczynowski et al., 2010; Starczynowski et al., 2011b), and is part of an MDS diagnostic miRNA signature (Sokol et al., 2011). Knockout of miR-146a results in an early onset of myeloid expansion in the marrow, and progression to more aggressive diseases such as lymphomas, marrow failure, and myeloid leukemia (Boldin et al., 2011; Zhao et al., 2011).

TRAF6 and IRAK1 are two immune-related targets of miR-146a (Starczynowski et al., 2010; Starczynowski et al., 2011a; Taganov et al., 2006), and as expected, miR-146a knockout mice have a dramatic increase in TRAF6 and IRAK1 protein within the hematopoietic compartment (Boldin et al., 2011; Zhao et al., 2011). TRAF6, a lysine (K)-63 E3 ubiquitin ligase, and IRAK1, a serine/threonine kinase, are interacting proteins and mediators downstream of Toll-like (TLR) and Interleukin-1 (IL1R) receptors. Activation of TLR or IL1R recruits a series of adaptor proteins resulting in phosphorylation of IRAK1 on Thr209. Phosphorylated IRAK1 binds to and activates TRAF6 resulting in NF-KB activation. Increasing clinical and biological data indicate that innate immune signaling is an important determinant of MDS pathophysiology (Bar et al., 2008; Chen et al., 2004; Hofmann et al., 2002; Vasikova et al., 2010).

Applicant has previously found the immune modulating kinase IRAK1 to be overexpressed and activated in MDS (Rhyasen & Starczynowski, B I C, 2014). Genetic or pharmacological inhibition of IRAK1 induces apoptosis and cell cycle arrest of MDS cells, and prolongs survival outcome in a human MDS xenograft model. IRAK1 has accordingly been found to be a drugable target in MDS.

As described herein, a global analysis of alternatively spliced genes and RNA isoforms in breast cancer, lung cancer, and AML was conducted and has revealed enrichment of alternatively spliced genes associated with inflammatory and immune pathways in cancerous/leukemic cells as compared to the respective normal cells/tissues. The global analysis of intron and exon usage in AML found the gene with the greatest differential cancer-specific isoform expression to be Interleukin Receptor Associated Kinase 4 (IRAK4), an innate immune signaling protein important for leukemic function.

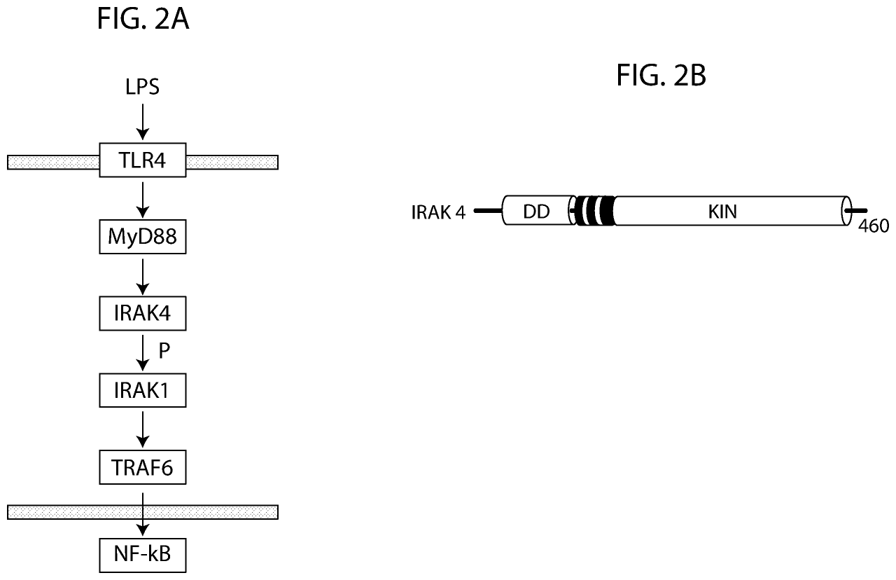

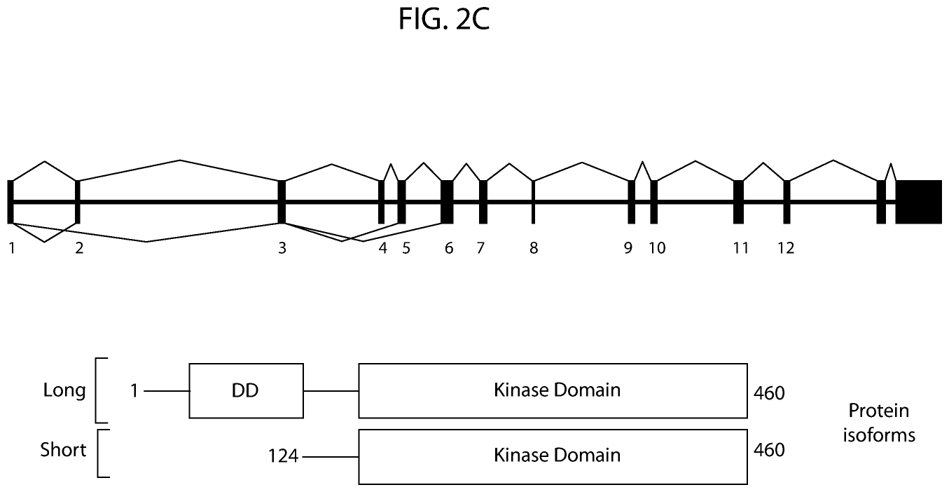

The interleukin-1 receptor-associated kinases (IRAKs) regulate intracellular signaling networks controlling inflammation and are expressed in many cell types. IRAKs also mediate signals from various cell receptors including toll-like receptors (TLRs). IRAK4 is a serine/threonine kinase which differs from and functions upstream of the other proteins in the IRAK family, such as IRAK1, IRAK2, and IRAKM, and Pelle. IRAK4 mediates signaling downstream of TLR signaling and consists of three functional domains, namely the death domain (DD, residues 1-100), the hinge domain (UD), and the kinase domain (residues 180-460), as shown in FIG. 2B; IRAK4 can include or exclude the N-terminal death domain to give one of two isoforms as a result of alternative splicing (the full sequence of IRAK4 can be found at Genbank, Accession No. AIC56391, Version AIC56391.1.). IRAK4 has been implicated in the pathogenesis of MDS/AML, and IRAK1/4 inhibitors have been shown to be effective in treating MDS, AML, and lymphoma (Rhyasen & Starczynowski, R I C, 2014). Knockdown of IRAK4 has been found to inhibit leukemic progenitor function. However, it has heretofore been unclear as to how to determine whether an IRAK1/4 inhibitor will be effective in a given subject with MDS/AML/lymphoma. There is a need for stratification of subjects into groups which can be treated effectively by a given treatment regimen.

Examination of the spliced IRAK4 isoforms by RNA sequencing showed that normal cells/tissues preferentially express an alternatively spliced IRAK4 isoform resulting from exclusion of the exon 4, which encodes a protein lacking the N-terminal death domain; this isoform is termed herein as “IRAK4-Short” or “IRAK4-S”. In contrast, many AML samples were found to show increased expression of an IRAK4 isoform that retains exon 4, encoding the full-length protein; this isoform is termed herein as “IRAK4-Long” or “IRAK4-L”. Skipping of IRAK4 exon 4 results in an in-frame deletion of the N-terminal death domain, which is required for IRAK4 oligomerization and efficient TLR signaling, while retaining the C-terminal kinase domain; accordingly, IRAK4 isoforms display differential signaling potential.

As described herein, cancer tissue has been found to express more IRAK4-L, while normal tissue expresses more IRAK4-S. Immunoblotting confirmed that MDS/AML samples predominantly express the IRAK4-Long protein, while normal hematopoietic BM cells express the IRAK4-Short protein lacking the N-terminal domain. Expression of IRAK4-L is correlated with worse prognosis.

Accordingly, some embodiments of the invention are directed to treating a subject having a disease or disorder, where the subject has enhanced IRAK4-Long expression and/or activity relative to IRAK4-Short, as compared to a normal control. In some embodiments, a normal control is a subject, or a sample from a subject, without the disease or disorder. In some embodiments, a normal control is a healthy subject, or a sample from a healthy subject. In some embodiments, a normal control is a subject who does not display symptoms of the disease or disorder, or a sample from a subject who does not display symptoms of the disease or disorder. In some embodiments, a normal control is a fixed, predetermined value based on a subject, or a sample from a subject, without the disease or disorder, or based on a healthy subject, or a sample from a healthy subject, or based on a subject who does not display symptoms of the disease or disorder, or a sample from a subject who does not display symptoms of the disease or disorder. In some embodiments of the invention, enhanced IRAK4-Long expression and/or activity relative to IRAK4-Short, as compared to a normal control, can be a ratio of IRAK4-Long to IRAK4-Short of greater than 0.50, greater than 0.60, greater than 0.65, greater than 0.70, greater than 0.75, greater than 0.80, greater than 0.85, greater than 0.90, greater than 0.95, greater than 1.0, greater than 1.10, greater than 1.20, greater than 1.30, greater than 1.40, greater than 1.50, greater than 1.60, greater than 1.70, greater than 1.80, greater than 1.90, greater than 2.00, greater than 2.25, greater than 2.50, greater than 2.75, greater than 3.0, greater than 3.5, or greater than 4.0.

IRAK4-Long expression is significantly associated with increased NF-kB and innate immune signaling and correlates with poor AML patient outcome. Functional characterization of the IRAK4 isoforms in human AML cell lines has revealed that IRAK4-Long induces NF-kB activation; in contrast, IRAK4-Short is less efficient at activating NF-kB (via phosphorylation of IKKbeta), yet it activates p38/MAPK signaling.

IRAK4 isoform expression and associated spliceosome gene mutations were examined in MDS/AML patients to analyze the alternative splicing regulation of IRAK4 exon 4. Of all somatic genetic mutations associated with AML, mutation of U2AF1 (S34F) was found to be significantly correlated with inclusion of exon 4 and expression of IRAK4-Long; accordingly, U2AF1 is involved with the splicing of IRAK4.

To explore the direct regulation of IRAK4 by U2AF1, wildtype or mutant (S34F) U2AF1 were expressed in CD34+ cord blood cells, and IRAK4 isoform expression and exon usage was determined by RNA-sequencing. Expression of U2AF1-S34F resulted in significant retention of IRAK4 exon 4 (i.e. IRAK4-Long), while expression of wildtype U2AF1 correlated with exclusion of IRAK4 exon 4 (i.e., IRAK4-Short). Examination of IRAK4 exon 4 usage in CD34+ cells from genetically-defined MDS patient samples revealed that nearly all MDS patient samples containing mutations in U2AF1 exhibited increased inclusion of exon 4 as compared to wild-type (WT) U2AF1 MDS samples or healthy controls. Higher expression of IRAK4-Long in MDS CD34+ cells or AML blasts was found to be associated with poor prognosis, and correlates with elevated blast counts and transfusion dependency in MDS. Utilizing a splicing reporter containing exon 4 and flanking intron sequences of IRAK4, overexpression of U2AF1-S34F induced retention of the cassette exon 4, while WT U2AF1 mediated exclusion of exon 4. Ectopic expression of U2AF1-S34F in AML cells resulted in significant retention of IRAK4 exon 4 and expression of IRAK4-Long protein. Mutations in U2AF1 therefore instruct expression of IRAK4 RNA isoforms with maximal functional potential.

Importantly, U2AF1-S34F AML cells were found to be more sensitive to pharmacologic inhibition of IRAK1/4 as compared to isogenic cells with WT U2AF1.

Taken together, these results illustrate the importance of cancer-associated RNA splicing alterations and their consequences on downstream molecular networks required for cancer pathogenesis. MDS and AML samples preferentially express IRAK4-Long, which is associated with increased innate immune signaling and poor AML patient outcome. Mutation of U2AF1 significantly correlates with inclusion of exon 4 and IRAK-Long protein expression, indicating that these mutations instruct expression of IRAK4 RNA isoforms. AML and MDS cells expressing IRAK4-Long or with mutations in U2AF1 are sensitive to IRAK inhibitors, particularly IRAK1/4 inhibitors. These findings demonstrate the importance of cancer-associated RNA splicing alterations and their consequences on downstream molecular networks required for cancer pathogenesis.

These results further demonstrate that mutations in U2AF1 induce expression of therapeutically targetable “active” IRAK4 isoforms and provide a genetic link to chronic innate immune signaling and IRAK1/4 activation in MDS and AML. U2AF1 mutations correlate to increased IRAK, as described herein, which leads to various cancers such as MDS and AML. Detection of U2AF1 mutations can therefore predict likelihood of success of IRAK inhibitors, such as IRAK1/4 inhibitors, as cancer treatment.

In one embodiment, detection of U2AF1 mutations in patients can be used to predict if a patient will express an IRAK isoform with inclusion of exon 4. In another embodiment, detection of U2AF1 mutations can be used to predict if a patient will have activated IRAK. In another embodiment, detection of U2AF1 mutations can be used to predict if a patient will express an IRAK isoform associated with increased NF-kB. In further embodiments, detection of U2AF1 mutations can be used to predict if an IRAK inhibitor can be used to treat a patient diagnosed with a disease associated with activated IRAK or increased NF-kB. In further embodiments, detection of U2AF1 mutations can be used to predict if an IRAK inhibitor can be used to treat a patient diagnosed with a MDS or AML associated with activated IRAK or increased NF-kB. In further embodiments, the U2AF1 mutation can be the S34F mutation.

It has been reported that between 5 and 11% of subjects having MDS have the S34F mutation in the U2AF1 gene (see, for example, Graubert et al., Nature Genetics, 2012, 44:53-7; Okeyo-Owuor et al., Leukemia, 2015, 29:909-17; Shirai et al., Blood, 2014, 124:827). Such patients can be expected to be treatable with an IRAK1/4 inhibitor. In contrast, MDS subjects who lack the U2AF1 mutation are less likely to benefit from administration of an IRAK1/4 inhibitor. Such patients may benefit from a different therapy which does not include an IRAK1/4 inhibitor.

Accordingly, it will be therapeutically effective to treat a subject with MDS or AML, who is found to have a mutation in U2AF1, with an IRAK1/4 inhibitor. For example, a subject with MDS or AML, who is found to have a mutation in U2AF1, can be treated with an IRAK1/4 inhibitor; in contrast, such an inhibitor can be expected to have reduced effectiveness in a subject with MDS or AML, who is found to express the wild-type U2AF1. For example, a subject with MDS or AML, who is found to have U2AF1-S34F mutation, can be treated with an IRAK1/4 inhibitor; in contrast, such an inhibitor can be expected to have reduced effectiveness in a subject with MDS or AML, who is found to express the wild-type U2AF1.

### Diseases and Disorders

Embodiments of the methods relate to administration of a compound or composition to treat any disease or disorder characterized by a U2AF1 mutation that enhances IRAK4-Long expression relative to IRAK4-Short.

In some embodiments, treating a disease or disorder disease or disorder characterized by a U2AF1 mutation that enhances IRAK4-Long expression relative to IRAK4-Short, such as MDS/AML/a type of cancer, can involve disease prevention, reducing the risk of the disease, ameliorating or relieving symptoms of the disease, eliciting a bodily response against the disease, inhibiting the development or progression of the disease, inhibiting or preventing the onset of symptoms of the disease, reducing the severity of the disease, causing a regression of the disease or a disease symptom, causing remission of the disease, preventing relapse of the disease, and the like. In some embodiments, treating includes prophylactic treatment. In some embodiments, treating does not include prophylactic treatment.

In some embodiments of the methods, the disease or disorder characterized by a U2AF1 mutation that enhances IRAK4-Long expression relative to IRAK4-Short can be MDS and/or AML and/or a type of cancer.

In some embodiments, the MDS can be selected from Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del(5q)-syndrome, chronic myelomonocytic leukemia (CMML), juvenile myelomonocytic leukemia (JMML), refractory cytopenia of childhood, or a combination thereof. In some embodiments, the MDS is primary MDS. In some embodiments, the MDS is secondary MDS.

In some embodiments, the AML can be selected from AML with recurrent genetic abnormalities (such as, for example, AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3, and the like), AML (megakaryoblastic) with a translocation between chromosomes 1 and 22, AML with myelodysplasia-related changes, AML related to previous chemotherapy or radiation (such as, for example, alkylating agent-related AML, topoisomerase II inhibitor-related AML, and the like), AML not otherwise categorized (does not fall into above categories—similar to FAB classification; such as, for example, AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis, and the like), myeloid sarcoma (also known as granulocytic sarcoma, chloroma or extramedullary myeloblastoma), undifferentiated and biphenotypic acute leukemias (also known as mixed phenotype acute leukemias), and the like.

In some embodiments, the type of cancer can be selected from breast cancer, cervical cancer, colorectal cancer, endometrial cancer, glioma, head and neck cancer, liver cancer, melanoma, ovarian cancer, pancreatic cancer, prostate cancer, renal cancer, stomach cancer, testicular cancer, thyroid cancer, urothelial cancer, and the like.

In some embodiments, the administration may decrease the incidence of one or more symptoms associated with MDS/AML/a type of cancer. In some embodiments, the administration may decrease marrow failure, immune dysfunction, transformation to overt leukemia, or combinations thereof in said individual, as compared to an individual not receiving said composition.

In some embodiments, the method may decrease a marker of viability of MDS cells or cancer cells. In one aspect, the method may decrease a marker of viability of MDS, AML, and/or cancer cells. The marker may be selected from survival over time, proliferation, growth, migration, formation of colonies, chromatic assembly, DNA binding, RNA metabolism, cell migration, cell adhesion, inflammation, or a combination thereof.

### IRAK Inhibitors

The present invention encompasses methods of treating a disease or disorder by administering a compound or composition comprising an IRAK inhibitor. In some embodiments, the IRAK inhibitor is an IRAK1/4 inhibitor.

Compounds and compositions which can be useful as IRAK inhibitors inhibitors are known in the art and are in development. Methods of treating a disease or disorder by administration of an IRAK inhibitor, such as an IRAK1/4 inhibitor, according to the present invention can involve any compound or composition which is demonstrated to inhibit IRAK, such as IRAK1 and/or IRAK 4. These include compounds which are currently commercially available, those which have been disclosed via publication, and those having yet to be contemplated. Methods of treating a disease or disorder by administration of an IRAK inhibitor which can be administered in conjunction with, or separately in a treatment course along with, one or more cancer treatments, as set forth herein.

IRAK inhibitors are known in the art. In some embodiments, the IRAK inhibitor can include, for example, one or more compounds such as N-acyl-2-aminobenzimidazoles, imidazo[1,2-a]pyridino-pyrimidines, imidazo[1,2-a]pyridino-pyridines, benzimidazolo-pyridines, N-(2-morpholinylethyl)-2-(3-nitrobenzoylamido)-benzimidazoles, 1-(2-(4-Morpholinyl)ethyl)-2-(3-nitrobenzoylamino)benzimidazoles, N-[1-[2-(4-Morpholinyl)ethyl]-1H-benzimidazol-2-yl]-3-nitrobenzamides, N-[ 1-(2-morpholin-4-ylethyl)benzimidazol-2-yl]-3-nitrobenzamides, N-[3-carbamoyl-1-(tetrahydro-2H-pyran-4-yl)-1H-pyrazol-4-yl]-2-(2-methylp-yridin-4-yl)-1,3-oxazole-4-carboxamides, N-[3-carbamoyl-1-(tetrahydro-2H-pyran-4-yl)-1H-pyrazol-4-yl]-2-(2-methylpyridin-4-yl)-1,3-oxazole-4-carboxamide hydrochlorides, 1-{[(2S)-5-oxopyrrolidin-2-yl]methoxy}-7-(propan-2-yloxy)isoquinoline-6-carboxamides, and the like, as well as derivatives such as pharmaceutically-acceptable salts, cocrystals, hydrates, solvates, or prodrugs thereof, and combinations thereof. In some embodiments, the IRAK inhibitor is R06245 or R00884. In some embodiments, the IRAK inhibitor may comprise an RNAi sufficient to inhibit IRAK1 expression.

Further IRAK inhibitors are disclosed in Buckley et al., Bioorganic & Medicinal Chemistry Letters 2008, 18(11):3211-3214; Buckley et al., Bioorganic & Medicinal Chemistry Letters 2008, 18(11):3291-3295; Buckley et al., Bioorganic & Medicinal Chemistry Letters 2008, 18(11):3656-3660; Chaudhary, et al., J. Med. Chem. 2015, 58, 96-110; Hynes and Nair, Annu. Rep. Med. Chem. 2014, 49, 117-133; W. Michael Seganish, Expert Opinion on Therapeutic Patents, 2016, 26:917-32; U.S. patent application Ser. No. 14/887,764, IRAK4 INHIBITORS AND USES THEREOF; U.S. patent application Ser. No. 15/110,309, INDAZOLE COMPOUNDS AS IRAK4 INHIBITORS; U.S. patent application Ser. No. 15/326,740, IRAK4 INHIBITING AGENTS; U.S. patent application Ser. No. 15/497,783, THERAPEUTIC TARGETING OF INTERLEUKIN-1 RECEPTOR-ASSOCIATED KINASE 4 (IRAK4) IN CANCERS CHARACTERIZED BY REARRANGEMENTS IN THE MIXED LINEAGE LEUKEMIA GENE (MLL-R); U.S. patent application Ser. No. 15/297,998, THERAPEUTIC COMBINATIONS OF AN IRAK4 INHIBITOR AND A BTK INHIBITOR; U.S. patent application Ser. No. 15/016,569, MACROCYCLIC COMPOUNDS AS IRAK1/4 INHIBITORS AND USES THEREOF; U.S. patent application Ser. No. 15/110,309, INDAZOLE COMPOUNDS AS IRAK4 INHIBITORS; U.S. patent application Ser. No. 14/661,656, HETEROCYCLIC COMPOUNDS AND USES THEREOF; U.S. patent application Ser. No. 13/738,410, IRAK INHIBITORS AND USES THEREOF; U.S. patent application Ser. No. 15/620,049, MACROCYCLIC COMPOUNDS AS IRAK1/4 INHIBITORS AND USES THEREOF; U.S. patent application Ser. No. 15/288,402, COMBINATION THERAPY FOR MDS; U.S. application Ser. No. 14/842,049, COMBINATION THERAPY FOR MDS; U.S. application Ser. No. 14/284,521, COMBINATION THERAPY FOR MDS; International Patent Application No. WO2017______, COMPOUNDS, COMPOSITIONS, METHODS FOR TREATING DISEASES, AND METHODS FOR PREPARING COMPOUNDS, filed on ______, 2017; and U.S. Provisional Application No. 62/248,050, COMPOUNDS, COMPOSITIONS, METHODS FOR TREATING DISEASES, AND METHODS FOR PREPARING COMPOUNDS, and elsewhere; it is contemplated that any such compound, or derivative, which is demonstrated or known in the art to have effectiveness as an IRAK1/4 inhibitor can be used in accordance with the present invention. All references listed above are incorporated herein with respect to their teachings of specific IRAK inhibiting compounds and genera.

In some embodiments, the IRAK inhibitor can be administered in combination with an agent selected from an apoptotic agent, an immune modulating agent, an epigenetic modifying agent, and combinations thereof.

In some embodiments, the method can involve administration of an apoptotic modulator. The apoptotic modulator comprises may comprise a BTK and/or a BCL2 inhibitor. BTK and BCL2 inhibitors are known in the art. In some embodiments, the method may comprise the step of administering to the individual an apoptotic modulator, wherein the apoptotic modulator may comprise a BCL2 inhibitor selected from ABT-263 (Navitoclax), ABT-737, ABT-199, GDC-0199, GX15-070 (Obatoclax), and combinations thereof, all available from Abbott Laboratories. In some embodiments, the method can involve administration of an immune modulator. The immune modulator can include, for example, Lenalidomide (Revlamid; Celgene Corporation). In some embodiments, the method can involve administration of an epigenetic modulator. The epigenetic modulator can include, for example, a hypomethylating agent such as azacitidine, decitabine, or a combination thereof.

### Cancer Treatments

Treatment regimens for various types of cancers can involve one or more elements selected from chemotherapy, targeted therapy, alternative therapy, immunotherapy, and the like.

Chemotherapy/Targeted Therapy/Alternative Therapy

Cancers are commonly treated with chemotherapy and/or targeted therapy and/or alternative therapy. Chemotherapies act by indiscriminately targeting rapidly dividing cells, including healthy cells as well as tumor cells, whereas targeted cancer therapies rather act by interfering with specific molecules, or molecular targets, which are involved in cancer growth and progression. Targeted therapy generally targets cancer cells exclusively, having minimal damage to normal cells. Chemotherapies and targeted therapies which are approved and/or in the clinical trial stage are known to those skilled in the art. Any such compound can be utilized in the practice of the present invention.

For example, approved chemotherapies include abitrexate (Methotrexate Injection), abraxane (Paclitaxel Injection), adcetris (Brentuximab Vedotin Injection), adriamycin (Doxorubicin), adrucil Injection (5-FU (fluorouracil)), afinitor (Everolimus), afinitor Disperz (Everolimus), alimta (PEMETREXED), alkeran Injection (Melphalan Injection), alkeran Tablets (Melphalan), aredia (Pamidronate), arimidex (Anastrozole), aromasin (Exemestane), arranon (Nelarabine), arzerra (Ofatumumab Injection), avastin (Bevacizumab), beleodaq (Belinostat Injection), bexxar (Tositumomab), BiCNU (Carmustine), blenoxane (Bleomycin), blincyto (Blinatumoma b Injection), bosulif (Bosutinib), busulfex Injection (Busulfan Injection), campath (Alemtuzumab), camptosar (Irinotecan), caprelsa (Vandetanib), casodex (Bicalutamide), CeeNU (Lomustine), CeeNU Dose Pack (Lomustine), cerubidine (Daunorubicin), clolar (Clofarabine Injection), cometriq (Cabozantinib), cosmegen (Dactinomycin), cotellic (Cobimetinib), cyramza (Ramucirumab Injection), cytosarU (Cytarabine), cytoxan (Cytoxan), cytoxan Injection (Cyclophosphamide Injection), dacogen (Decitabine), daunoXome (Daunorubicin Lipid Complex Injection), decadron (Dexamethasone), depoCyt (Cytarabine Lipid Complex Injection), dexamethasone Intensol (Dexamethasone), dexpak Taperpak (Dexamethasone), docefrez (Docetaxel), doxil (Doxorubicin Lipid Complex Injection), droxia (Hydroxyurea), DTIC (Decarbazine), eligard (Leuprolide), ellence (Ellence (epirubicin)), eloxatin (Eloxatin (oxaliplatin)), elspar (Asparaginase), emcyt (Estramustine), erbitux (Cetuximab), erivedge (Vismodegib), erwinaze (Asparaginase Erwinia chrysanthemi), ethyol (Amifostine), etopophos (Etoposide Injection), eulexin (Flutamide), fareston (Toremifene), farydak (Panobinostat), faslodex (Fulvestrant), femara (Letrozole), firmagon (Degarelix Injection), fludara (Fludarabine), folex (Methotrexate Injection), folotyn (Pralatrexate Injection), FUDR (FUDR (floxuridine)), gazyva (Obinutuzumab Injection), gemzar (Gemcitabine), gilotrif (Afatinib), gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Ifex (Ifosfamide), Imbruvica (Ibrutinib), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Keytruda (Pembrolizumab Injection), Kyprolis (Carfilzomib), Lanvima (Lenvatinib), Leukeran (Chlorambucil), Leukine (Sargramostim), Leustatin (Cladribine), Lonsurf (Trifluridine and Tipiracil), Lupron (Leuprolide), Lupron Depot (Leuprolide), Lupron DepotPED (Leuprolide), Lynparza (Olaparib), Lysodren (Mitotane), Marqbo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Odomzo (Sonidegib), Oncaspar (Pegaspargase), Oncovin (Vincristine), Ontak (Denileukin Diftitox), onxol (Paclitaxel Injection), opdivo (Nivolumab Injection), panretin (Alitretinoin), paraplatin (Carboplatin), perj eta (Pertuzumab Injection), platinol (Cisplatin), platinol (Cisplatin Injection), platinolAQ (Cisplatin), platinolAQ (Cisplatin Injection), pomalyst (Pomalidomide), prednisone Intensol (Prednisone), proleukin (Aldesleukin), purinethol (Mercaptopurine), reclast (Zoledronic acid), revlimid (Lenalidomide), rheumatrex (Methotrexate), rituxan (Rituximab), roferonA alfaa (Interferon alfa-2a), rubex (Doxorubicin), sandostatin (Octreotide), sandostatin LAR Depot (Octreotide), soltamox (Tamoxifen), sprycel (Dasatinib), sterapred (Prednisone), sterapred DS (Prednisone), stivarga (Regorafenib), supprelin LA (Histrelin Implant), sutent (Sunitinib), sylatron (Peginterferon Alfa-2b Injection (Sylatron)), sylvant (Siltuximab Injection), synribo (Omacetaxine Injection), tabloid (Thioguanine), taflinar (Dabrafenib), tarceva (Erlotinib), targretin Capsules (Bexarotene), tasigna (Decarbazine), taxol (Paclitaxel Injection), taxotere (Docetaxel), temodar (Temozolomide), temodar (Temozolomide Injection), tepadina (Thiotepa), thalomid (Thalidomide), theraCys BCG (BCG), thioplex (Thiotepa), TICE BCG (BCG), toposar (Etoposide Injection), torisel (Temsirolimus), treanda (Bendamustine hydrochloride), trelstar (Triptorelin Injection), trexall (Methotrexate), trisenox (Arsenic trioxide), tykerb (lapatinib), unituxin (Dinutuximab Injection), valstar (Valrubicin Intravesical), vantas (Histrelin Implant), vectibix (Panitumumab), velban (Vinblastine), velcade (Bortezomib), vepesid (Etoposide), vepesid (Etoposide Injection), vesanoid (Tretinoin), vidaza (Azacitidine), vincasar PFS (Vincristine), vincrex (Vincristine), votrient (Pazopanib), vumon (Teniposide), wellcovorin IV (Leucovorin Injection), xalkori (Crizotinib), xeloda (Capecitabine), xtandi (Enzalutamide), yervoy (Ipilimumab Injection), yondelis (Trabectedin Injection), zaltrap (Ziv-aflibercept Injection), zanosar (Streptozocin), zelboraf (Vemurafenib), zevalin (Ibritumomab Tiuxetan), zoladex (Goserelin), zolinza (Vorinostat), zometa (Zoledronic acid), zortress (Everolimus), zydelig (Idelalisib), zykadia (Ceritinib), zytiga (Abiraterone), and the like, in addition to analogs and derivatives thereof. For example, approved targeted therapies include ado-trastuzumab emtansine (Kadcyla), afatinib (Gilotrif), aldesleukin (Proleukin), alectinib (Alecensa), alemtuzumab (Campath), axitinib (Inlyta), belimumab (Benlysta), belinostat (Beleodaq), bevacizumab (Avastin), bortezomib (Velcade), bosutinib (Bosulif), brentuximab vedotin (Adcetris), cabozantinib (Cabometyx [tablet], Cometriq [capsule]), canakinumab (Ilaris), carfilzomib (Kyprolis), ceritinib (Zykadia), cetuximab (Erbitux), cobimetinib (Cotellic), crizotinib (Xalkori), dabrafenib (Tafinlar), daratumumab (Darzalex), dasatinib (Sprycel), denosumab (Xgeva), dinutuximab (Unituxin), elotuzumab (Empliciti), erlotinib (Tarceva), everolimus (Afinitor), gefitinib (Iressa), ibritumomab tiuxetan (Zevalin), ibrutinib (Imbruvica), idelalisib (Zydelig), imatinib (Gleevec), ipilimumab (Yervoy), ixazomib (Ninlaro), lapatinib (Tykerb), lenvatinib (Lenvima), necitumumab (Portrazza), nilotinib (Tasigna), nivolumab (Opdivo), obinutuzumab (Gazyva), ofatumumab (Arzerra, HuMax-CD20), olaparib (Lynparza), osimertinib (Tagrisso), palbociclib (Ibrance), panitumumab (Vectibix), panobinostat (Farydak), pazopanib (Votrient), pembrolizumab (Keytruda), pertuzumab (Perj eta), ponatinib (Iclusig), ramucirumab (Cyramza), rapamycin, regorafenib (Stivarga), rituximab (Rituxan, Mabthera), romidepsin (Istodax), ruxolitinib (Jakafi), siltuximab (Sylvant), sipuleucel-T (Provenge), sirolimus, sonidegib (Odomzo), sorafenib (Nexavar), sunitinib, tamoxifen, temsirolimus (Torisel), tocilizumab (Actemra), tofacitinib (Xeljanz), tositumomab (Bexxar), trametinib (Mekinist), trastuzumab (Herceptin), vandetanib (Caprelsa), vemurafenib (Zelboraf), venetoclax (Venclexta), vi smodegib (Erivedge), vorinostat (Zolinza), ziv-aflibercept (Zaltrap), and the like, in addition to analogs and derivatives thereof.

Those skilled in the art can determine appropriate chemotherapy and/or targeted therapy and/or alternative therapy options, including treatments that have been approved and those that in clinical trials or otherwise under development. Some targeted therapies are also immunotherapies. Any relevant chemotherapy, target therapy, and alternative therapy treatment strategies can be utilized, alone or in combination with one or more additional cancer therapy, in the practice of the present invention.

Immunotherapy

In some embodiments, immunotherapies include cell-based immunotherapies, such as those involving cells which effect an immune response (such as, for example, lymphocytes, macrophages, natural killer (NK) cells, dendritic cells, cytotoxic T lymphocytes (CTL), antibodies and antibody derivatives (such as, for example, monoclonal antibodies, conjugated monoclonal antibodies, polyclonal antibodies, antibody fragments, radiolabeled antibodies, chemolabeled antibodies, etc.), immune checkpoint inhibitors, vaccines (such as, for example, cancer vaccines (e.g. tumor cell vaccines, antigen vaccines, dendritic cell vaccines, vector-based vaccines, etc.), e.g. oncophage, sipuleucel-T, and the like), immunomodulators (such as, for example, interleukins, cytokines, chemokines, etc.), topical immunotherapies (such as, for example, imiquimod, and the like), injection immunotherapies, adoptive cell transfer, oncolytic virus therapies (such as, for example, talimogene laherparepvec (T-VEC), and the like), immunosuppressive drugs, helminthic therapies, other non-specific immunotherapies, and the like. Immune checkpoint inhibitor immunotherapies are those that target one or more specific proteins or receptors, such as PD-1, PD-L1, CTLA-4, and the like. Immune checkpoint inhibitor immunotherapies include ipilimumab (Yervoy), nivolumab (Opdivo), pembrolizumab (Keytruda), and the like. Non-specific immunotherpaies include cytokines, interleukins, interferons, and the like. In some embodiments, an immunotherapy assigned or administered to a subject can include an interleukin, and/or interferon (IFN), and/or one or more suitable antibody-based reagent, such as denileukin diftitox and/or administration of an antibody-based reagent selected from the group consisting of ado-trastuzumab emtansine, alemtuzumab, atezolizumab, bevacizumab, blinatumomab, brentuximab vedotin, cetuximab, catumaxomab, gemtuzumab, ibritumomab tiuxetan, ilipimumab, natalizumab, nimotuzumab, nivolumab, ofatumumab, panitumumab, pembrolizumab, rituximab, tositumomab, trastuzumab, vivatuxin, and the like. In some embodiments, an immunotherapy assigned or administered to a subject can include an indoleamine 2,3-dioxygenase (IDO) inhibitor, adoptive T-cell therapy, virotherapy (T-VEC), and/or any other immunotherapy whose efficacy extensively depends on anti-tumor immunity.

Those skilled in the art can determine appropriate immunotherapy options, including treatments that have been approved and those that in clinical trials or otherwise under development. Any relevant immunotherapy treatment strategies, alone or in combination with one or more additional cancer therapy, can be utilized in the practice of the present invention.

Other Cancer Treatments

In addition to chemotherapies, targeted therapies, alternative therapies, and immunotherapies, cancer can additionally be treated by other strategies. These include surgery, radiation therapy, hormone therapy, stem cell transplant, precision medicine, and the like; such treatments and the compounds and compositions utilized therein are known to those skilled in the art. Any such treatment strategies can be utilized in the practice of the present invention.

Alternative treatment strategies have also been used with various types of cancers. Such treatment can be used alone or in combination with any other treatment modality. These include exercise, massage, relaxation techniques, yoga, acupuncture, aromatherapy, hypnosis, music therapy, dietary changes, nutritional and dietary supplements, and the like; such treatments are known to those skilled in the art. Any such treatment strategies can be utilized, alone or in combination with one or more additional cancer therapy, in the practice of the present invention.

### Administration

Particular aspects of the invention relate to the use of cancer treatments, in the form of therapeutic compounds and/or compositions, directly administered to a subject. Such compounds and/or compositions and/or their physiologically acceptable salts or esters, can be used for the preparation of a medicament (pharmaceutical preparation). They can be converted into a suitable dosage form together with at least one solid, liquid and/or semiliquid excipient or assistant and, if desired, in combination with one or more further active ingredients.

Therapeutic compounds and/or compositions can be prepared and administered in a wide variety of ophthalmic, oral, parenteral, and topical dosage forms. The therapeutic compounds and/or compositions can be administered by eye drop. Also, therapeutic compounds and/or compositions can be administered by injection (e.g. intravenously, intramuscularly, intravitreally, intracutaneously, subcutaneously, intraduodenally, or intraperitoneally). As such, therapeutic compounds and/or compositions can also be administered by intravitreal injection. Also, therapeutic compounds and/or compositions can be administered by inhalation, for example, intranasally. Additionally, therapeutic compounds and/or compositions can be administered transdermally. It is also envisioned that multiple routes of administration (e.g., intramuscular, oral, ocular) can be used to administer therapeutic compounds and/or compositions.

### Formulations

Particular aspects of the invention furthermore include medicaments comprising at least one therapeutic compound or composition suitable for treatment of cancer, and/or its pharmaceutically usable derivatives, solvates and stereoisomers, including mixtures thereof in all ratios, and optionally excipients and/or assistants.

According to particular aspects, the therapeutic compounds and compositions can be administered by any conventional method available for use in conjunction with pharmaceutical drugs, either as individual therapeutic agents or in a combination of therapeutic agents. Such therapeutics can be administered by any pharmaceutically acceptable carrier, including, for example, any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional medium or agent is incompatible with the active compound, such media can be used in the compositions of the invention. Supplementary active compounds can also be incorporated into the compositions. A pharmaceutical composition in particular aspects of the invention is formulated to be compatible with its intended route of administration. Routes of administration include for example, but are not limited to, intravenous, intramuscular, and oral, and the like. Additional routes of administration include, for example, sublingual, buccal, parenteral (including, for example, subcutaneous, intramuscular, intraarterial, intradermal, intraperitoneal, intracisternal, intravesical, intrathecal, or intravenous), transdermal, oral, transmucosal, and rectal administration, and the like.

Solutions or suspensions used for appropriate routes of administration, including, for example, but not limited to parenteral, intradermal, or subcutaneous application, and the like, can include, for example, the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates, or phosphates and agents for the adjustment of tonicity such as sodium chloride or dextrose, and the like. The pH can be adjusted with acids or bases, such as, for example, hydrochloric acid or sodium hydroxide, and the like. The parenteral preparation can be enclosed in, for example, ampules, disposable syringes, or multiple dose vials made of glass or plastic, and the like.

Exemplary pharmaceutical compositions suitable for injectable use include, for example, sterile aqueous solutions (where water soluble) or dispersions and sterile powders for the extemporaneous preparation of sterile injectable solutions or dispersion, and the like. For intravenous administration, suitable carriers include, for example, physiological saline, bacteriostatic water, Cremophor ELTM (BASF, Parsippany, N.J.) or phosphate buffered saline (PBS), and the like. In all cases, the composition should be fluid to the extent that easy syringability exists. The carrier can be a solvent or dispersion medium containing, for example, water, ethanol, polyol (for example, glycerol, propylene glycol, and liquid polyethylene glycol, and the like), and suitable mixtures thereof, and the like. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, such as, for example, parabens, chlorobutanol, phenol, ascorbic acid, thimerosal, and the like. In many cases, it can be preferable to include isotonic agents, such as, for example, sugars, polyalcohols such as mannitol, sorbitol, and sodium chloride, and the like, in the composition. Prolonged absorption of the injectable compositions can be brought about by including in the composition an agent which delays absorption such as, for example, aluminum monostearate and gelatin, and the like.

Exemplary sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Exemplary oral compositions generally include an inert diluent or an edible carrier. They can be enclosed in gelatin capsules or compressed into tablets, for example. For oral administration, the agent can be contained in enteric forms to survive the stomach or further coated or mixed to be released in a particular region of the gastrointestinal (GI) tract by known methods. For the purpose of oral therapeutic administration, the active compound can be incorporated with excipients and used in the form of tablets, troches, or capsules, or the like. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following exemplary ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring, or the like. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatin, carbohydrates, such as lactose or starch, magnesium stearate, talc or VASELINE®. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders or also as nasal sprays. The novel compounds may also be lyophilized and the resultant lyophilizates used, for example, to prepare injection preparations. The preparations indicated may be sterilized and/or comprise assistants, such as lubricants, preservatives, stabilizers and/or wetting agents, emulsifying agents, salts for modifying the osmotic pressure, buffer substances, colorants and flavors and/or a plurality of further active ingredients, for example one or more vitamins.

For administration by inhalation, the compositions can be delivered in the form of an aerosol spray from pressured container or dispenser, which contains a suitable propellant, e.g., a gas such as carbon dioxide, or a nebulizer, or the like. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives, and the like. Transmucosal administration can be accomplished through the use of nasal sprays or suppositories. For transdermal administration, the active compounds are formulated into ointments, salves, gels, or creams as generally known in the art.

The compositions can also be prepared in the form of suppositories (e.g., with conventional suppository bases such as cocoa butter and other glycerides) or retention enemas for rectal delivery.

In particular embodiments, therapeutic compounds and/or compositions are prepared with carriers that will protect the compound against rapid elimination from the body, such as a controlled release formulation, including implants and microencapsulated delivery systems, and the like. Biodegradable, biocompatible polymers can be used, such as, for example, ethylene vinyl acetate, polyanhydrides, polyglycolic acid, collagen, polyorthoesters, and polylactic acid, and the like. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, which is incorporated herein by reference in its entirety.

In some embodiments, therapeutic compounds and/or compositions can be prepared in liquid pharmaceutical compositions for ocular administration. The composition for ocular use can contain one or more agents selected from the group of buffering agents, solubilizing agents, coloring agents, viscosity enhancing agents, and preservation agents in order to produce pharmaceutically elegant and convenient preparations.

In some embodiments, the composition for ocular use can contain preservatives for protection against microbiological contamination, including but not limited to benzalkodium chloride and/or EDTA. Other possible preservatives include but are not limited to benzyl alcohol, methyl parabens, propyl parabens, and chlorobutanol. Preferably, a preservative, or combination of preservatives, will be employed to impart microbiological protection in addition to protection against oxidation of components.

In some embodiments, therapeutic compounds and/or compositions can be administered orally as tablets, aqueous or oily suspensions, lozenges, troches, powders, granules, emulsions, capsules, syrups or elixirs. The composition for oral use can contain one or more agents selected from the group of sweetening agents, flavoring agents, coloring agents and preserving agents in order to produce pharmaceutically elegant and palatable preparations. Accordingly, there are also provided pharmaceutical compositions comprising a pharmaceutically acceptable carrier or excipient and one or more therapeutic compounds and/or compositions.

In some embodiments, tablets contain the acting ingredient in admixture with non-toxic pharmaceutically acceptable excipients that are suitable for the manufacture of tablets. These excipients can be, for example, (1) inert diluents, such as calcium carbonate, lactose, calcium phosphate, carboxymethylcellulose, or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets can be uncoated or coated by known techniques to delay disintegration and absorption in the gastrointestinal tract and thereby provide a sustained action over a longer period. For example, a time delay material such as glyceryl monostearate or glyceryl di stearate can be employed.

For preparing pharmaceutical compositions from therapeutic compounds and/or compositions, pharmaceutically acceptable carriers can be either solid or liquid. Solid form preparations include powders, tablets, pills, capsules, cachets, suppositories, and dispersible granules. A solid carrier can be one or more substance that can also act as diluents, flavoring agents, binders, preservatives, tablet disintegrating agents, or an encapsulating material.

A compound disclosed herein, in the form of a free compound or a pharmaceutically-acceptable pro-drug, metabolite, analogue, derivative, solvate or salt, can be administered, for in vivo application, parenterally by injection or by gradual perfusion over time. Administration can be intravenously, intraperitoneally, intramuscularly, subcutaneously, intracavity, or transdermally. For in vitro studies the compounds can be added or dissolved in an appropriate biologically acceptable buffer and added to a cell or tissue.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for therapeutic compounds and/or compositions are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The therapeutic compounds and/or compositions can also be incorporated into liposomes or administered via transdermal pumps or patches. Pharmaceutical admixtures suitable for use in the pharmaceuticals compositions and methods disclosed herein include those described, for example, in PHARMACEUTICAL SCIENCES (17th Ed., Mack Pub. Co., Easton, Pa.) and WO 96/05309, the teachings of both of which are hereby incorporated by reference.

In some embodiments, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, growth factors and inert gases and the like.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations can contain, in addition to the active component, colorants, flavors, stabilizers, buffers, artificial and natural sweeteners, dispersants, thickeners, solubilizing agents, and the like.

Some compounds can have limited solubility in water and therefore can require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions can be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions disclosed herein can additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

There are provided various pharmaceutical compositions useful for ameliorating certain diseases and disorders. The pharmaceutical compositions according to one embodiment are prepared by formulating a compound disclosed herein in the form of a free compound or a pharmaceutically-acceptable pro-drug, metabolite, analogue, derivative, solvate or salt, either alone or together with other pharmaceutical agents, suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers.

Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See e.g., Goodman and Gilman (eds.), 1990, THE PHARMACOLOGICAL BASIS FOR THERAPEUTICS (7th ed.).

The pharmaceutical compositions are preferably prepared and administered in dose units. Solid dose units are tablets, capsules and suppositories. For treatment of a subject, depending on activity of the compound, manner of administration, nature and severity of the disease or disorder, age and body weight of the subject, different daily doses can be used.

Under certain circumstances, however, higher or lower daily doses can be appropriate. The administration of the daily dose can be carried out both by single administration in the form of an individual dose unit or else several smaller dose units and also by multiple administrations of subdivided doses at specific intervals.

The method by which the compound disclosed herein can be administered for oral use would be, for example, in a hard gelatin capsule wherein the active ingredient is mixed with an inert solid diluent, or soft gelatin capsule, wherein the active ingredient is mixed with a co-solvent mixture, such as PEG 400 containing Tween-20. A compound disclosed herein can also be administered in the form of a sterile injectable aqueous or oleaginous solution or suspension. The compound can generally be administered intravenously or as an oral dose of 0.1 μg to 20 mg/kg given, for example, every 3-12 hours.

Formulations for oral use can be in the form of hard gelatin capsules wherein the active ingredient is mixed with an inert solid diluent, for example, calcium carbonate, calcium phosphate or kaolin. They can also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients can be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which can be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions can be in the form of a sterile injectable aqueous or oleagenous suspension. This suspension can be formulated according to known methods using those suitable dispersing or wetting agents and suspending agents that have been mentioned above. The sterile injectable preparation can also a sterile injectable solution or suspension in a non-toxic parenterally-acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

A compound disclosed herein can also be administered in the form of ophthalmic compositions applied topically to the eye, preferably in the form of eye drops. A compound disclosed herein can also be administered in the form of intravitreal injection.

A compound disclosed herein can also be administered in the form of suppositories for rectal administration of the drug. These compositions can be prepared by mixing the drug with a suitable non-irritating excipient that is solid at ordinary temperature but liquid at the rectal temperature and will therefore melt in the rectum to release the drug. Such materials include cocoa butter and polyethylene glycols.

The therapeutic compounds and/or compositions as used in the methods disclosed herein can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

For topical use, creams, ointments, jellies, solutions or suspensions, etc., containing therapeutic compounds and/or compositions, are employed.

In addition, some treatment compounds can form solvates with water or common organic solvents. Such solvates are encompassed within the scope of the methods contemplated herein.

Dosage

The pharmaceutical compositions contemplated herein can be administered locally or systemically in a therapeutically effective dose. Amounts effective for this use will, of course, depend on the severity of the disease or disorder and the weight and general state of the subject. Typically, dosages used in vitro can provide useful guidance in the amounts useful for in situ administration of the pharmaceutical composition, and animal models can be used to determine effective dosages for treatment of particular disorders.

Various considerations are described, e. g., in Langer, 1990, Science, 249: 1527; Goodman and Gilman's (eds.), 1990, Id., each of which is herein incorporated by reference and for all purposes. Dosages for parenteral administration of active pharmaceutical agents can be converted into corresponding dosages for oral administration by multiplying parenteral dosages by appropriate conversion factors. As to general applications, the parenteral dosage in mg/mL times 1.8=the corresponding oral dosage in milligrams (“mg”). As to oncology applications, the parenteral dosage in mg/mL times 1.6=the corresponding oral dosage in mg. An average adult weighs about 70 kg. See e.g., Miller-Keane, 1992, ENCYCLOPEDIA & DICTIONARY OF MEDICINE, NURSING & ALLIED HEALTH, 5th Ed., (W. B. Saunders Co.), pp. 1708 and 1651.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The details for the dosage unit forms of the invention are dictated by and directly dependent on the unique characteristics of the active compound and the particular therapeutic effect to be achieved, and the limitations inherent in the art of compounding such an active compound for the treatment of individuals. Such details are known to those of skill in the art.

The dosage administered will, of course, vary depending upon known factors, such as the pharmacodynamic characteristics of the particular agent and its mode and route of administration; the age, health, sex, weight, and diet of the recipient; the nature and extent of the symptoms; the kind of concurrent treatment; the time and frequency of treatment; the excretion rate; and the effect desired.

Therapeutically effective amounts for use in humans can be determined from animal models. For example, a dose for humans can be formulated to achieve a concentration that has been found to be effective in animals. The dosage in humans can be adjusted by monitoring enzymatic inhibition and adjusting the dosage upwards or downwards, as described above.

Dosages can be varied depending upon the requirements of the patient and the compound being employed. The dose administered to a patient, in the context of the methods disclosed herein, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. The composition can, if desired, also contain other compatible therapeutic agents.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

A daily dosage of active ingredient can be expected to be about 0.001 to 1000 milligrams (mg) per kilogram (kg) of body weight, with the preferred dose being 0.01 to about 30 mg/kg. The quantity of active component in a unit dose preparation can be varied or adjusted from 0.1 mg to 10000 mg, more typically 1.0 mg to 1000 mg, most typically 10 mg to 500 mg, according to the particular application and the potency of the active component. In some embodiments of a method disclosed herein, the dosage range is 0.001% to 10% w/v. In some embodiments, the dosage range is 0.1% to 5% w/v.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Materials, Methods, and General Experimental Procedures

Aberrant Isoform Variance Vs. Cumulative Variance

For every gene, cumulative and isoform variances, as well as lowest isoform correlations were calculated. Cumulative variance: the variance in the cumulative expression (sum of all isoforms), isoform variance: the average variance in the expression of individual isoforms, lowest isoform correlation: the most negative correlation between all pairs of isoforms for the gene. Negative value in the latter indicates isoform switching as a mechanism of regulation of that gene's expression.

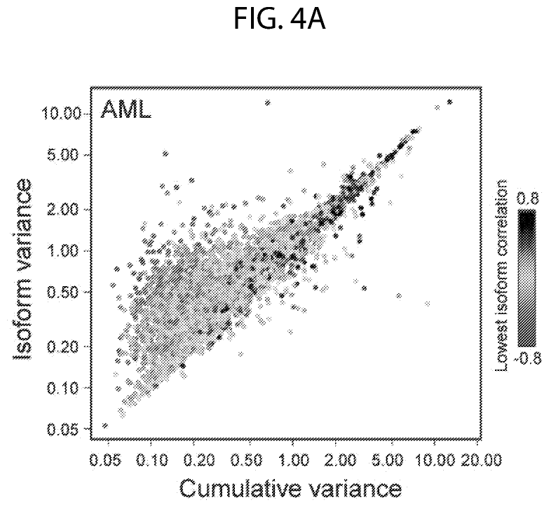

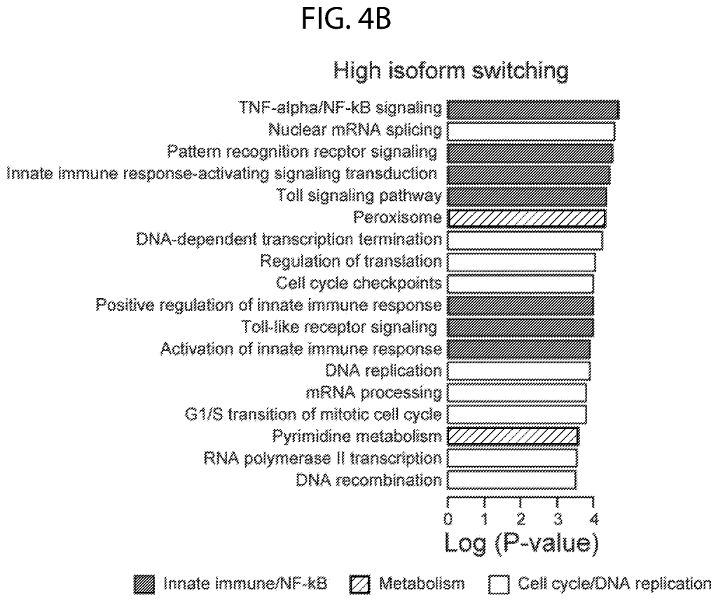

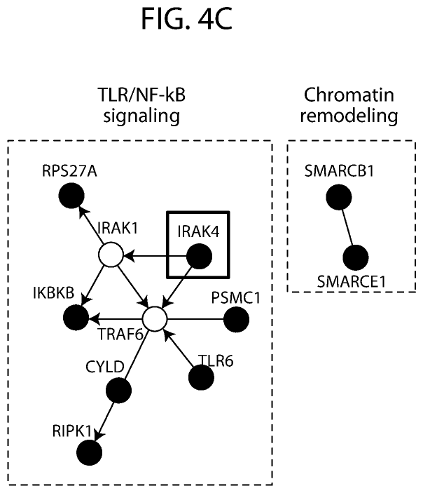

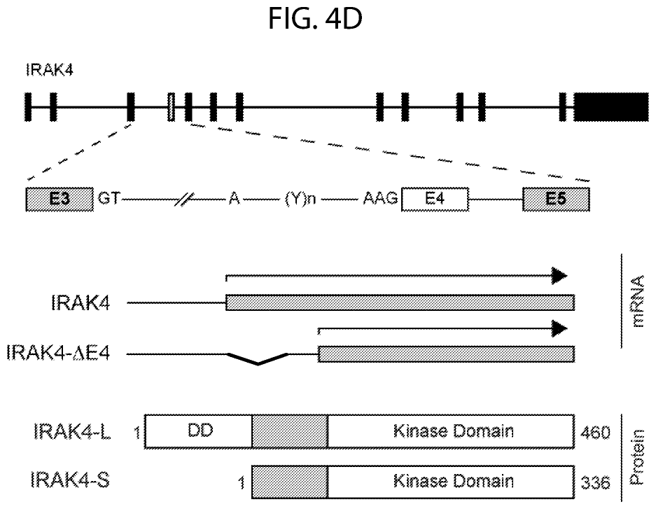

Enriched Pathway Analysis (FIG. 4B)

Pathway enrichment (hypergeometric distribution) of genes that are regulated at the level of isoform switching.

Cell Lines and CD34+ Cells

Acute myeloid leukemic cell lines, HL60, THP1, and TF-1 were purchased from the American Type Culture Collection. The myelodysplastic cell line, MDS-L, was provided by Dr. Kaoru Tohyama (Kawasaki Medical School, Okayama, Japan) (Matsuoka et al., 2010). Cell-lines were cultured in RPMI 1640 medium with 10% FBS and 1% penicillin-streptomycin. Additionally, both the MDSL and TF-1 cell lines were cultured in the presence of 10 ng/mL human recombinant IL-3 (Stem Cell Technologies, Tukwila, Wash.). Human CD34+ umbilical cord blood and adult marrow-derived mononuclear cells were obtained from the Translational Research Development Support Laboratory of Cincinnati Children's Hospital under an approved Institutional Review Board protocol. Human CD34+ UCB cells and primary MDS/AML cells were maintained in StemSpan Serum-Free Expansion Media (Stem Cell Technologies) supplemented with 10 ng/mL of recombinant human stem cell factor (SCF), Flt3 ligand (Flt3L), thrombopoietin (TPO), IL-3, and IL-6 (Stem Cell Technologies).

RT-PCR and qRT-PCR

Total RNA was extracted and purified using Quick-RNA MiniPrep (Zymo Research, Irvine, Calif., R1055) and reverse transcription was carried out using SuperScript cDNA Synthesis Kit (Invitrogen, Carlsbad, Calif.). Quantitative PCR was performed with Taqman Master Mix (Life Technologies, Carlsbad, Calif.) for TNFAIP3 (Hs00234713_m1), IL-6 (Hs99999032_m1), and GAPDH (Hs02758991_g1). qPCR was performed on an Applied Biosystems StepOne Plus Real-Time PCR System (Applied Biosystems, Foster City, Calif.).

TABLE 1
Primers utilized.

| Primers | Forward | Reverse |
|---|---|---|
| IRAK4 | 5′-gctgcctcaatgttggacta-3′ | 5′-tctggacttgaggagtcagg-3′ |
| | (SEQ ID NO: 1) | (SEQ ID NO: 2) |
| CXCL2 | 5′-cgcccatggttaagaaaatca-3′ | 5′-ccttctggtcagttggatttgc-3′ |
| | (SEQ ID NO: 3) | (SEQ ID NO: 4) |
| CXCL8 | 5′-cttggcagccttcctgattt-3′ | 5′-ttctttagcactccttggcaaaa-3′ |
| | (SEQ ID NO: 5) | (SEQ ID NO: 6) |

Immunoblotting

Total protein lysates were obtained from cells by lysing the samples in cold RIPA buffer (50 mM Tris-HCl, 150 mM NaCl, 1 mM EDTA, 1% Triton X-100 and 0.1% SDS), in the presence of PMSF, sodium orthovanadate, protease and phosphatase inhibitors. Protein concentration was evaluated by a BCA assay (32106, Pierce, Rockford, Ill.). The bound proteins were separated by SDS-polyacrylamide gel electrophoresis, transferred to nitrocellulose membranes, and analyzed by immunoblotting. Western blot analysis was performed with the following antibodies: IRAK4 C-term (ab5985; Abcam, Cambridge, UK), IRAK N-term (4363; Cell Signaling Technology), GAPDH (5174; Cell Signaling Technology), Flag (F3165; Sigma, St. Louis, Mo.), IRAK1 (sc-7883; Santa Cruz, Dallas, Tex.), phospho-IRAK1 (T209) (A1074; AssaybioTech, Sunnyvale, Calif.), U2AF1 (ab86305; Abeam), IKKβ (2370; Cell Signaling Technology), phospho-IKKα/β (2697; Cell Signaling Technology), p38 MAPK (9212; Cell Signaling Technology), phopho-p38 MAPK (4631; Cell Signaling Technology), ERK (4695; Cell Signaling Technology), phospho-ERK (4377; Cell Signaling Technology), p65 (8242; Cell Signaling Technology) and phospho-p65 (3033; Cell Signaling Technology).

shRNA-Mediated Knockdown of IRAK4

The pLKO.1 constructs were obtained from the Lentiviral core at CCHMC. Puromycin resistance gene was replaced by green fluorescent protein (GFP). Two independent and validated pLKO.1-shIRAK4 constructs were obtained: TRCN000000063 and TRCN000000065.

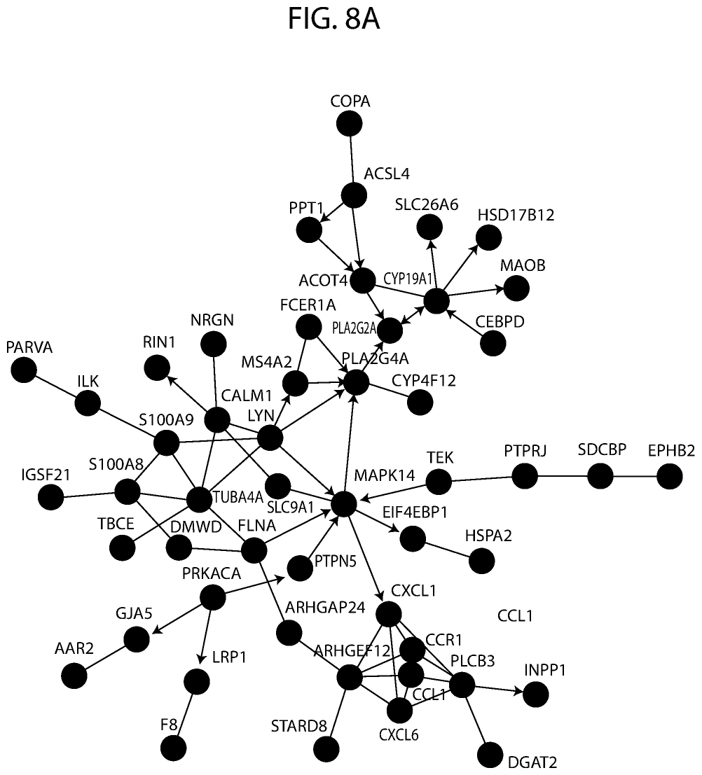

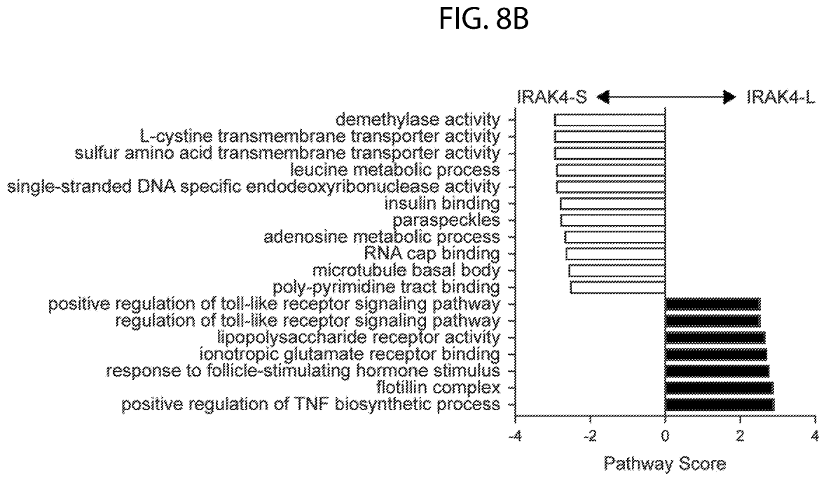

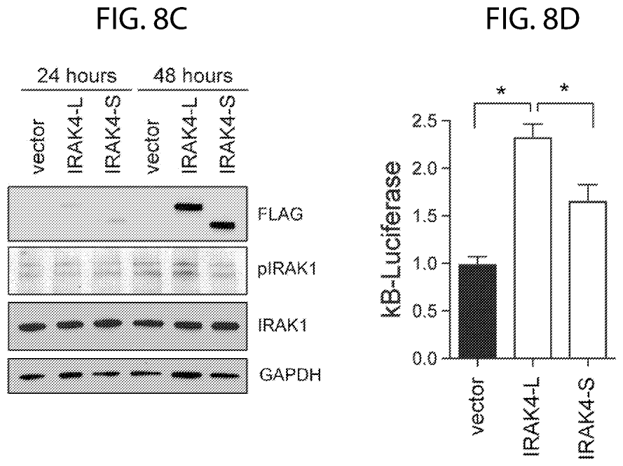

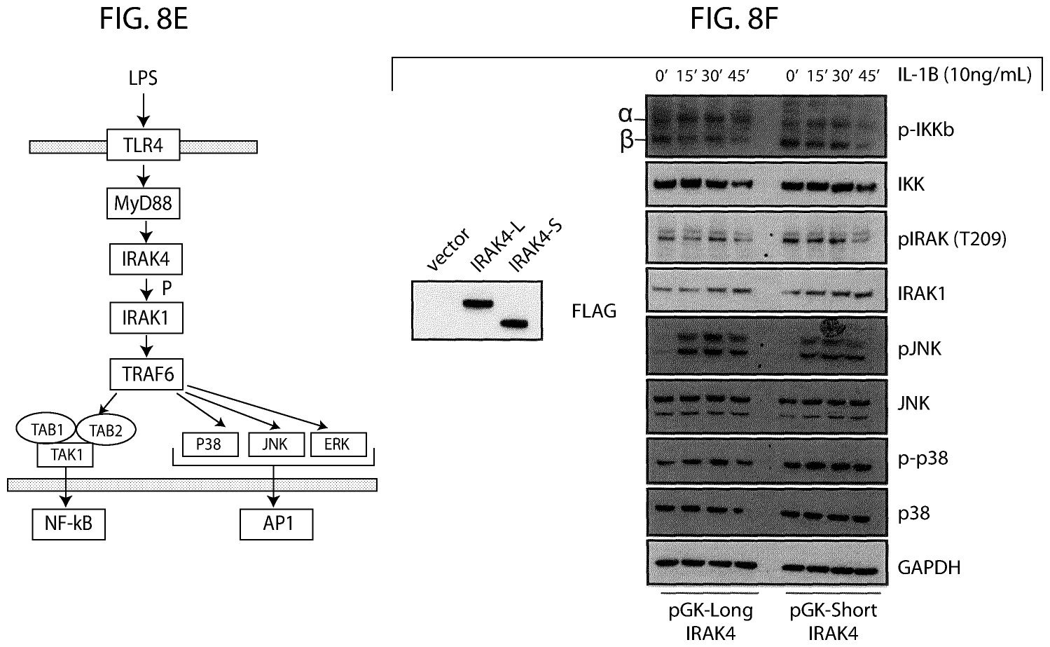

Pathway Analysis (FIG. 8B)

Pathway over-representation profiles of genes that correlate with the expression of the short or the long isoform expression of IRAK4 in TCGA AML dataset. Pathway scores were calculated using NetWalker (Komurov et al, BMC Genomics 2012).

Plasmids and Transfection

IRAK4-Long and IRAK4-Short cDNA were created by custom gene synthesis (IDT). FLAG tags were inserted in frame at the 5′-end of human IRAK4. FLAG-IRAK4 was cloned into pcDNA3.0 with EcoRI and BamHI, and into pMSCV-pGK-GFP with EcoRI and XhoI. Transfections were performed using TransIT-LT1 transfection reagent (Minis MIR 2306).

Luciferase Assay

HEK293 cells were transfected for 24-48 h with KB-luciferase and TK Renilla plasmids along with IRAK4-Long or IRAK4-Short. Lysates were analyzed for NF-KB reporter activity using the dual luciferase reporter assay system (Promega, Madison, Wis., E1910). Retro- and lentiviral transduction of cells has been previously described (Fang et al., 2014).

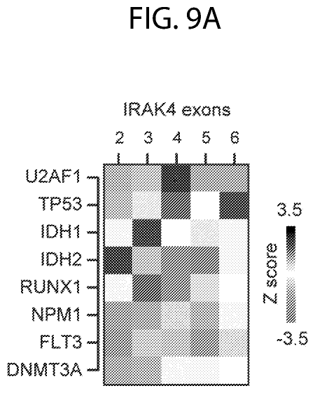

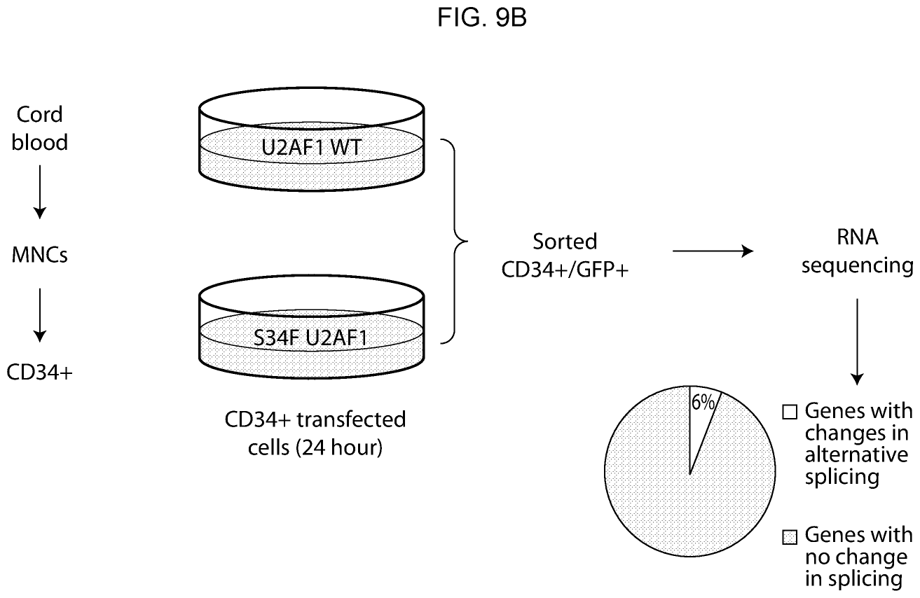

Mutation Analysis (FIG. 9A)

Z-score heatmap for the partial correlations of each exon's expression with the indicated mutations in AML.

Creation of Inducible U2AF1(S34F) and U2AF1(WT) Cell Lines

Doxycycline-inducible FLAG-tagged WT U2AF1 or FLAG-tagged mutant U2AF1(S34F) lentiviral expression plasmids have been previously described, and lentivirus was generated in 293T cells with the packaging plasmids pMD-G, pMD-Lg and REV. Concentrated virus (multiplicity of infection (MOI) of 3 for K562, MOI of 5 for OCI-AML3) was used to transduce K562 cells (ATCC, CCL243) or OCI-AML3 (DSMZ, ACC 582). Transduced cells (marked by green fluorescent protein) were isolated by flow cytometry cell sorting. Expression of mutant or WT U2AF1 was induced using the indicated concentrations of doxycycline hyclate (Sigma) in water. (Shirai C, et al., 2017)

Reporter Cell Lines

IRAK4 exon 4 and its flanking intronic sequences (50 nucleotides upstream of and 50 nucleotides downstream from exon 4) were inserted into the EcoRI and BamHI sites of pflareA vectors. pflareA-exon4 vectors were linearized by DraIII and transfected into 293T cells. Cell colonies stably expressing the reporter were selected by 1 mg/mL G418 and stable expression of GFP and RFP.

Reagents

The IRAK1 inhibitor (IRAK1/4 inhibitor or IRAK-Inh; Amgen Inc., Bothell, Wash.) was purchased from Sigma-Aldrich (15409). Recombinant human IL1-□ was purchased from PeproTech (Rocky Hill, N.J., 200-01B).

Growth Curves

Trypan blue exclusion was measured using an automated cell counter (BioRad, Hercules, Calif., TC10). Cells were replenished with fresh media, doxycycline and drug every fifth day.

Clonogenic Progenitor Assays

Clonogenic progenitor frequencies were determined by plating 1×103 K562 cells/ml in methylcellulose (Methocult H4236; Stem Cell Technologies). Colonies were scored after 7 days.

### Example 2

### Differential Isoform Expression in Cancerous Cells

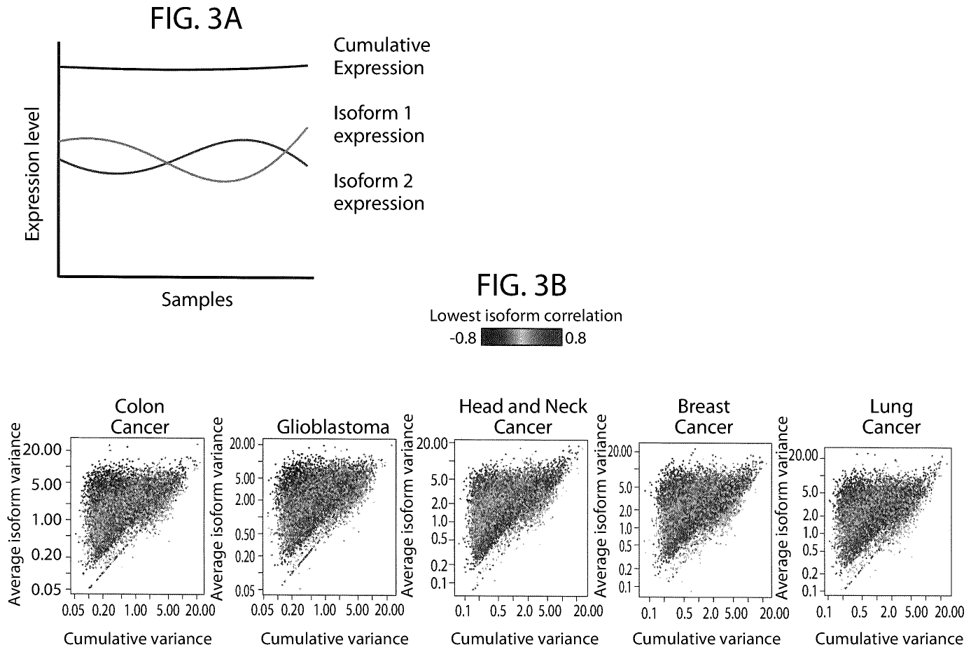

Alternative splicing of mRNA transcripts results in the production of multiple gene isoforms, which typically code for proteins with divergent sequences, domains, and functions. Aberrant RNA splicing has been implicated in the pathogenesis of certain human cancers, as shown in FIG. 3. In cancer cells, gene isoform usage frequently differs from that of normal, non-transformed cells of the same tissue, perhaps contributing to their malignant character. Accordingly, many genes have increased susceptibility to changes in isoform expression. This includes genes that are involved in innate immune signaling. One such gene is IRAK4, which undergoes alternative splicing that encodes two protein isoforms.

A subset of alternatively spliced genes were found to exhibit changes in AML and are involved in innate immune signaling, as shown in FIG. 4. Variance in gene isoform expression was measured relative to cumulative variance in gene expression in a publically available datatset of AML samples to identify the genes undergoing the greatest variability in isoform usage. Pathway analysis of the genes undergoing significant isoform variability revealed several innate immune signaling pathways, including TNF-alpha/NF-kB signaling.

Among the most differentially spliced genes in the dataset was IRAK4, a serine/threonine kinase that mediates signaling downstream of the toll-like receptor (TLR) superfamily and upstream of multiple inflammatory signaling pathways, including MAPK and NF-kB. Further analysis revealed that IRAK4 is among the genes with the most variable isoform usage across all cancer types. Full-length IRAK4 (IRAK4-Long) protein possesses three functional domains: the death domain, a hinge domain, and a kinase domain. The death domain is involved in protein oligimerization, important for signal transduction downstream of TLR activation. Alternative splicing of IRAK4 transcripts results in the retention or removal of exon 4, which encodes a portion of the death domain. When exon 4 is skipped, an in-frame deletion of the death domain occurs and a shorter isoform of IRAK4 (IRAK4-Short) that lacks the death domain is produced.

FIG. 4A shows the cumulative variance vs isoform variance in AML. FIG. 4B shows the pathways associated with genes that undergo the greatest changes in AML isoform switching. FIG. 4C shows the genes involved in two of these pathways, namely TLF/NF-kB signaling and chromatin remodeling. FIG. 4D shows the two IRAK4 variants within the coding region encode two protein isoforms, Long (IRAK4-L) and Short (IRAK4-S). The spliced variants of IRAK4 either include exon 4 or have it skipped in subsets of human AML. The short protein isoform lacks the N-terminal death domain while retaining its kinase domain. Expression of IRAK4-L is associated with poor survival.

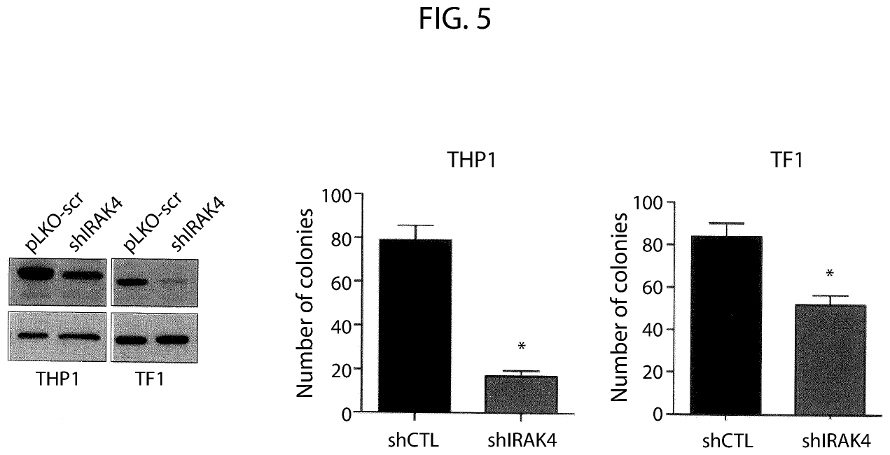

Knockdown of IRAK4 was found to inhibit leukemic progenitor function, as shown in FIG. 5, which shows immunoblotting and reduced colonies following knockdown of IRAK4 in THP-1 and TF1 leukemic cells.

### Example 3

### IRAK4 Isoform Expression in AML

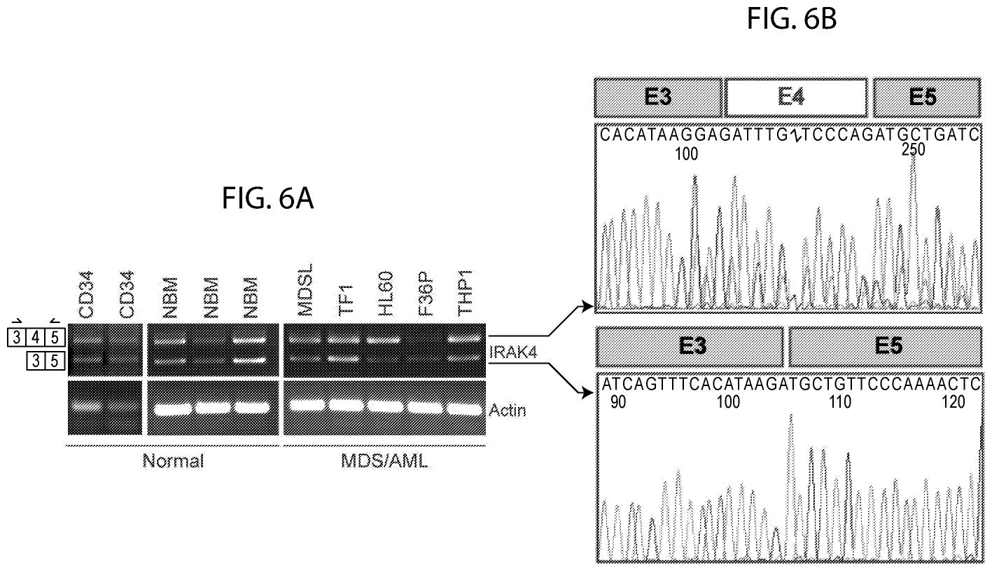

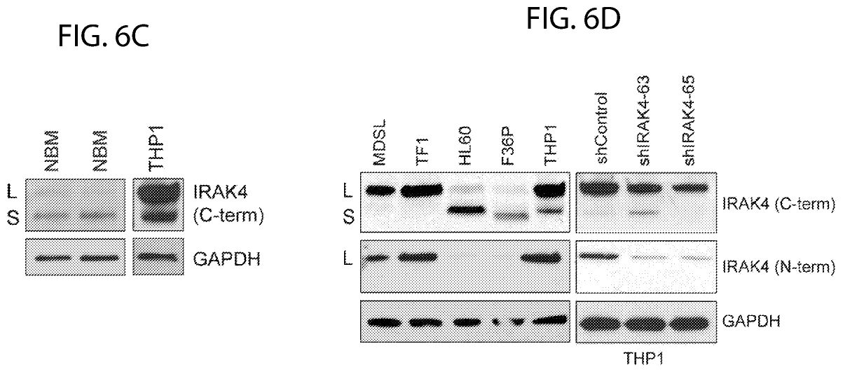

Subsequent experiments were conducted to validate the observations from the RNA-sequencing that two IRAK4 isoforms are expressed in AML samples. IRAK4 mRNA variants were found to encode two protein isoforms in AML, as shown in FIG. 6. While both isoforms are expressed in normal bone marrow and MDS/AML cell lines, long IRAK4 protein isoform is expressed higher relative to the short IRAK4 isoform in AML cells lines; in contrast, short IRAK4 protein isoform is expressed higher relative to the long IRAK4 isoform in normal bone marrow. Expression of the long IRAK4 isoform is associated with survival.

RT-PCR was performed using primers flanking exon 4 in CD34+ cells, NBM, and MDS/AML cell lines and detected both isoforms. The identity of the isoforms was confirmed by Sanger sequencing. A C-terminal specific antibody that detects both protein isoforms was utilized to confirm that two IRAK4 RNA isoforms are expressed as proteins in MDS and AML cell lines and in normal bone marrow. shRNAs were utilized that target either the long IRAK4 transcripts only (shIRAK4 63) or all the IRAK4 transcripts (shIRAK4 65) to confirm that the observed protein isoforms are formed by mRNAs of the expected splicing pattern.

FIG. 6A shows results of PCT amplification of MDS, AML and normal bone marrow (NBM) samples for IRAK4-L or IRAK4-S based on the presence or absence of exon 4 and distinguished by size using primers in exon 3 and 5. FIG. 6B shows that the IRAK4 exon 4 cassette was confirmed by sequencing. FIGS. 6C and 6D show results from immunoblotting NBM and MDS/AML cell lines for expression of long and short IRAK4 isoforms. Knockdown of the IRAK4 isoforms in THP1 cells was validated by immunoblotting (right).

### Example 4

### IRAK4 Isoform Expression Patterns in Normal Vs Cancerous Cells

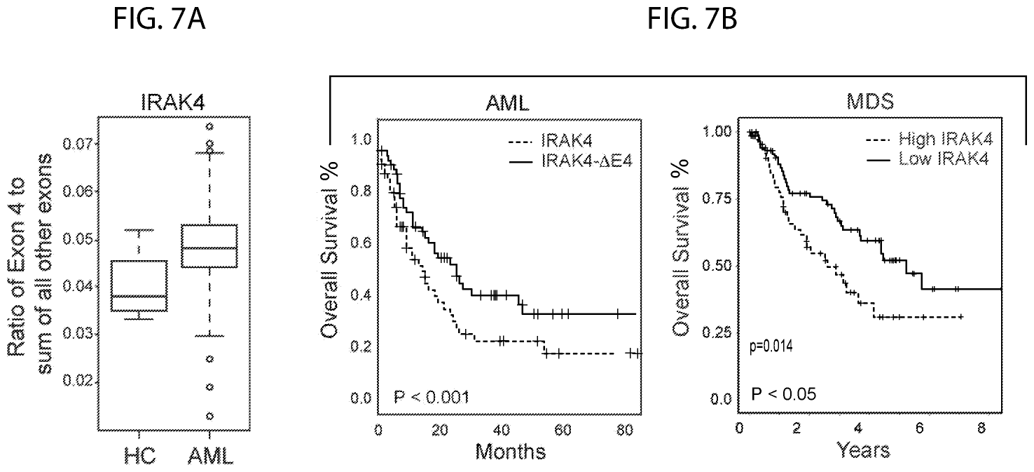

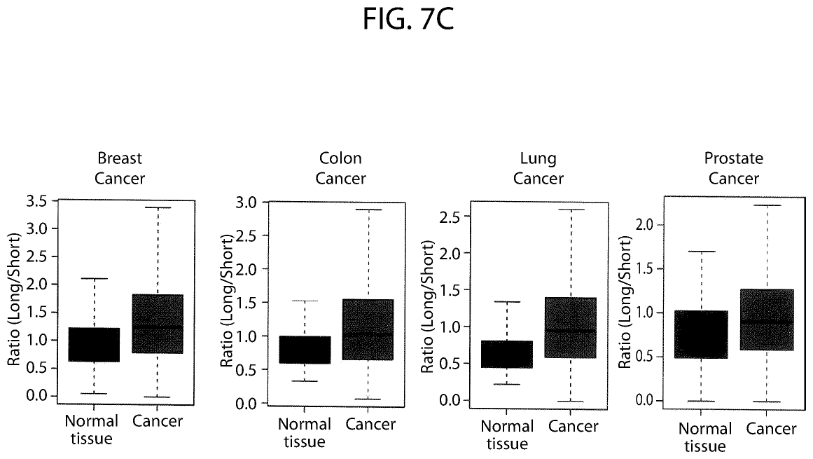

Next, the expression of these isoforms was evaluated in RNA-sequencing data sets from TCGA to address whether these isoform patterns differ between normal and cancer. Inclusion of IRAK4 exon 4 was found to be associated with AML, as shown in FIG. 7. Tumors were found to express more IRAK4-Long mRNA relative to short, while normal tissue expresses more IRAK4-Short mRNA relative to long. To determine whether increased IRAK4-Long expression in cancer contributes to disease severity, AML and MDS patients were stratified based on IRAK4 isoform expression, as measured by the presence of exon 4. Patients with expression of full-length IRAK4 were found to be associated with worse patient outcome. Taken together, these data indicate that the IRAK4-Long isoform is expressed preferentially in AML and contributes to disease progression.

FIG. 7A shows the ratio of IRAK4-Long to IRAK4-Short isoform expression from the Cancer Genome Atlas. FIG. 7B shows the Kaplan-Meier curve of AML and MDS patients stratified based on IRAK4 isoform expression (as measured by presence of exon 4 in AML or by expression of the long isoform in MDS). FIG. 7C shows the differential expression of IRAK4 mRNA variants in various human cancers, including AML.

### Example 5

### Effect of IRAK4-Long Expression

To determine the effect of IRAK4-Long expression on gene expression, gene expression profiles of patients that have higher expression of IRAK4-Long versus the IRAK4-Short were examined. IRAK4-Long protein was found to exhibit maximal activation of innate immune and NF-kB signaling, as shown in FIG. 8. The IRAK4-Long protein was found to be functionally involved in immune regulation. To test experimentally whether the IRAK4-Long isoform differentially activates NF-kB, flag-tagged Long or Short IRAK4 was over-expressed in 293T cells, and assessed phospho-IRAK1 expression, the immediate downstream signaling event of IRAK4 activation. IRAK4-Long was able to activate IRAK1 while IRAK4-Short was not. Similarly, overexpression of IRAK4-Long was found to significantly increase the activity of NF-Kb reporter compared to IRAK4-Short.

FIG. 8A shows several gene networks that were found to be enriched in IRAK4-L expressing AML patients. FIG. 8B shows a pathway analysis depicting enriched pathways in AML patients with high expression of IRAK4-L relative to IRAK4-S, and high expression of IRAK4-S relative to IRAK4-L. FIG. 8C shows results from immunoblotting 293T cells transfected with FLAG-IRAK4-L or FLAG-IRAK4-S using the indicated antibodies. FIG. 8D shows NF-kB reporter activity (kB-Luciferase) measured in 293T cells transfected with FLAG-IRAK4-L or FLAG-IRAK4-S. FIGS. 8E and 8F shows that long IRAK4 differentially regulates NF-kB and MAPK as compared to IRAK4-S.

### Example 6

### U2AF1 Mutations in AML and MDS

Subsequent research examined whether IRAK4-Long expression correlates with the presence of any genetic alterations in AML. To do this, available mutation data were examined for correlation with increased IRAK4 exon 4 retention. U2AF1 mutations are observed in MDS and AML. This involves binding the 3′ AG splice acceptor dinucleotide of the pre-mRNA target intron required for spliceosome activation. Inclusion of the IRAK4 exon 4 is associated with U2AF1 mutations in AML and MDS, as shown in FIG. 9.

Exon 4 retention was found to be correlated with U2AF1 mutation. U2AF1 is a splicing factor that is mutated in MDS and AML. In normal cells, it functions by binding to the 3 prime AG splice acceptor site in the pre-mRNA intron. U2AF1 mutation confers a change in function. As further verification that U2AF1 is associated with exon 4 retention, a Sashimi plot of mRNA sequencing samples of CD34+ cells isolated from the bone marrow of MDS patients has been generated, wherein samples with mutated U2AF1 displayed increased retention of IRAK4 exon 4, thereby expressing more IRAK4-Long (data not shown). RT-PCR analysis of patient bone marrow samples quantified by densitometry showed that mutation in U2AF1, but not splicing factor SF3B1, was associated with significantly higher exon 4 retention compared to healthy controls and MDS patients with no mutation in splicing factors (data not shown).

FIG. 9A shows that genetic alterations were found to be correlated with IRAK4 exon 4 retention in AML. FIG. 9B shows the experimental design for determining U2AF1's control of splicing.

### Example 7

### U2AF1-S34F Mutation Contributes to IRAK4 EXON 4 Retention

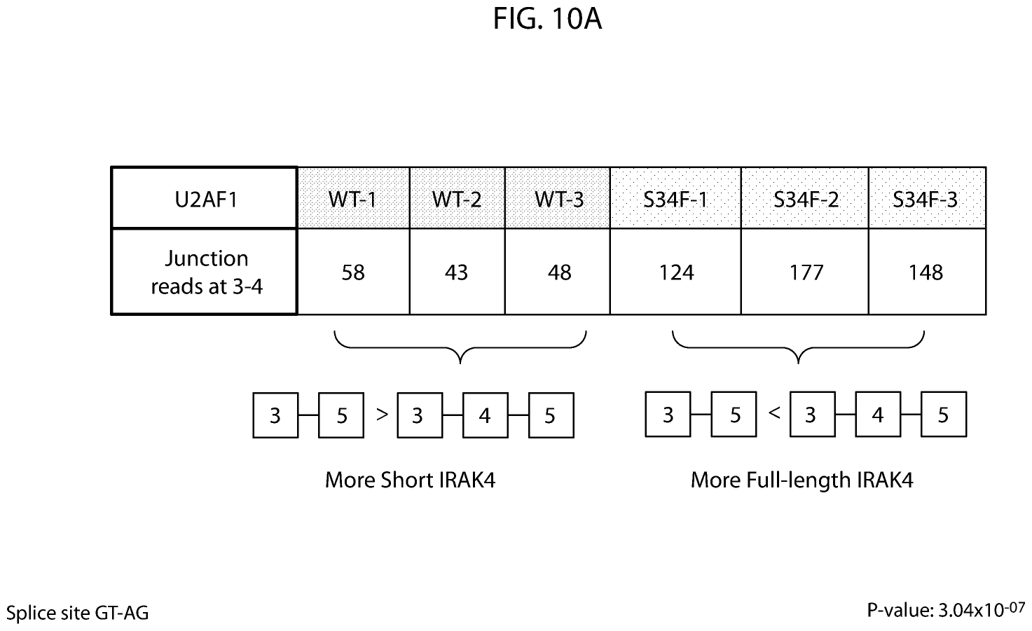

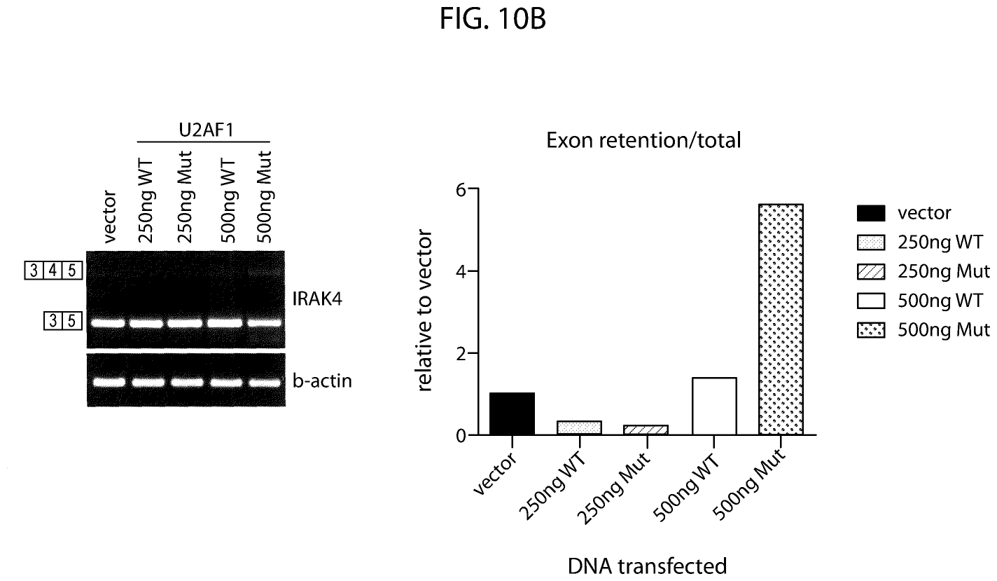

To test mechanistically if U2AF1 contributes to IRAK4 exon 4 retention, RNA-sequencing was performed on normal CD34+ cells isolated from cord blood and transfected with WT- or S34F-U2AF1 to examine splicing changes. The U2AF1-S34F mutation was found to significantly correlate with IRAK4 exon 4 retention in CD34+U2AF1 expressing cells, as shown in FIG. 10. FIG. 10A shows that an increased amount of IRAK4-S is present with the wild-type U2AF1 gene, whereas a significantly increased amount of IRAK4-L is present with the U2AF1-S34F mutation. FIG. 10B shows that the mutant U2AF1 confers increased exon retention.

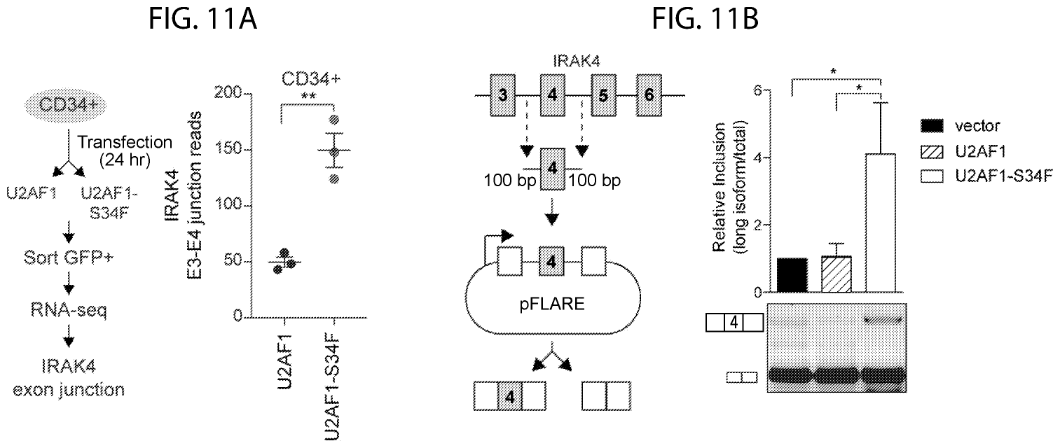

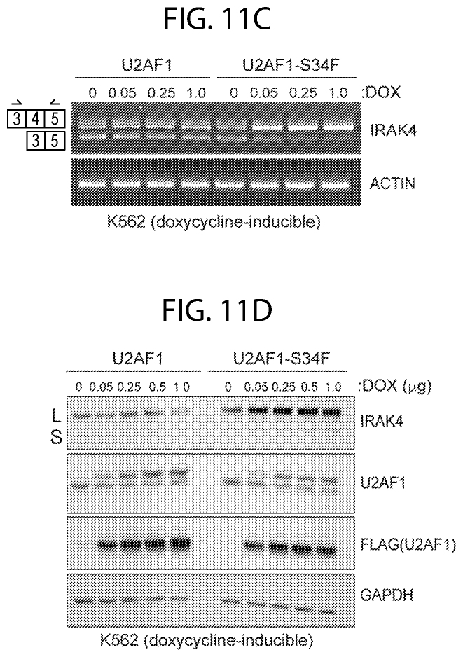

U2AF1-S34F was found to directly regulate inclusion of IRAK4 exon 4 and expression of IRAK4-Long protein, as shown in FIG. 11. Within this data set, there was a higher number of junction reads at the IRAK4 exon 3-4 junction in the S34F-U2AF1 cells compared to WT-U2AF1 cells, suggesting that U2AF1 mutation is significantly correlated with IRAK4 exon 4 retention. To determine whether U2AF1 mutation affects IRAK4 exon 4 retention, a dual-fluorescence reporter was utilized to differentiate exon splicing from exon retention (Stoilov, 2008). Either WT- or S34F-U2AF1 plasmids were transfected in 293T cell lines stably expressing the reporter containing IRAK4 exon 4 and measured exon inclusion or exclusion by PCR. Expression of S34F-U2AF1 resulted in greater inclusion of IRAK4 exon 4. Moreover, the experiments utilized a leukemia cell line, K562, expressing lentiviral plasmids that inducibly express FLAG-tagged WT- or S34F-U2AF1. When cells were treated with increasing amounts of doxycycline, it was found that ectopic expression of mutant U2AF1 results in IRAK4-Long expression in a dose dependent manner at both the RNA and protein level.

FIG. 11A shows the experimental design used to measure RNA splicing changes in human CD34+ cells transduced with WT or mutant U2AF1. RNA-sequencing junction reads for IRAK4 exon 3-4 are shown. FIG. 11B shows the results from transfecting 293T cells expressing an IRAK4 exon 4 minigene cassette with WT or mutant U2AF1 and PCR amplified for exon retention (top band) and exon exclusion (bottom band). Bar graph represents intensity of top PCR band over total intensity relative to vector. FIGS. 11C and 11D show that K562 cells express FLAG-U2AF1 or FLAG-U2AF1-S34F under the control of a doxycycline (DOX)-inducible promoter. For FIG. 11C, IRAK4 exon 4 usage was determined by RT-PCR. For FIG. 11D, IRAK4 and U2AF1 protein expression was determined by immunoblotting.

### Example 8

### U2AF1 Mutation Activates Innate Immune Pathway

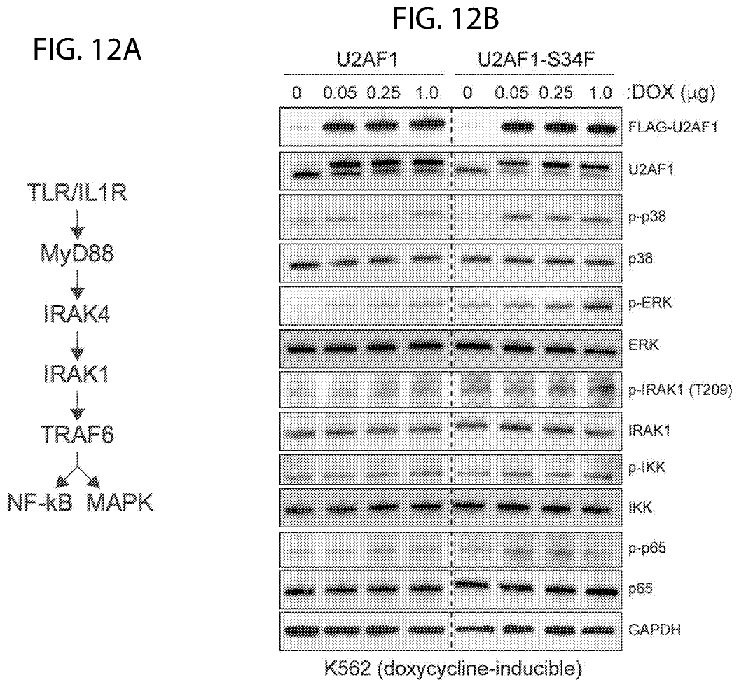

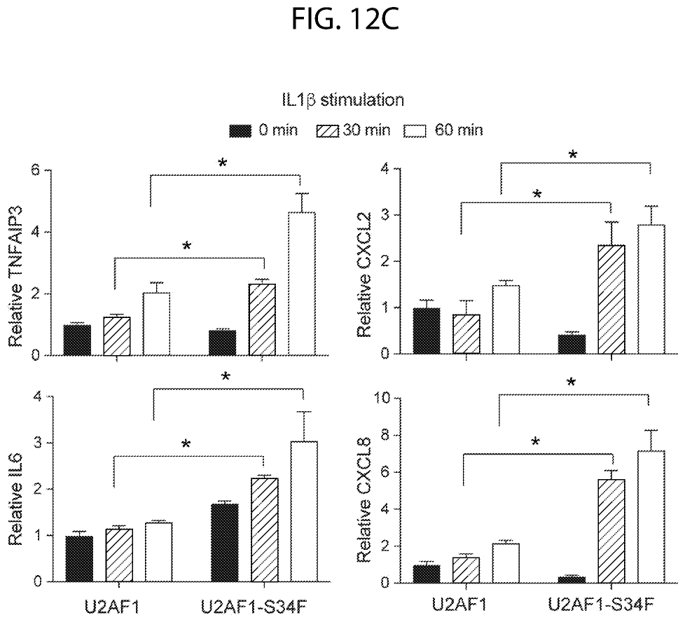

U2AF1-mutant AML cells were found to exhibit increased innate immune pathway activation, as shown in FIG. 12. Since IRAK4 activation is upstream of NF-kB and MAPK signaling pathways, subsequent experiments assessed whether U2AF1 mutation affected these signaling pathways. K562-U2AF1 cells were treated with increasing concentrations of doxycycline and NF-kB and MAPK activation was assessed. Expression of phospho-p38 and phospho-ERK were found to be elevated when U2AF1 is mutated compared to WT U2AF1. Similarly, when mutant U2AF1 was induced, there was a strong increase in phospho-IRAK1 (T209), phospho-IKK and phospho-p65 expression, indicating increased NF-kB and MAPK activation via mutation of U2AF1. Consistent with this data, S34F-U2AF1 cells were found to display increased expression of NF-kB target genes compared to WT upon stimulation. Taken together, these data indicate that U2AF1 mutation activates NF-kB and MAPK pathways.

FIG. 12A provides an overview of innate immune signaling. FIG. 12B shows results from immunoblotting K562 cells expressing wild-type or mutant U2AF1 using the indicated antibodies. FIG. 12C shows results from stimulating K562 cells expressing wild-type or mutant U2AF1 with IL-1β and examination for NF-kB target genes by qRT-PCR.

### Example 9

### U2AF1-Mutant Cells have Increased Sensitivity to IRAK Inhibition

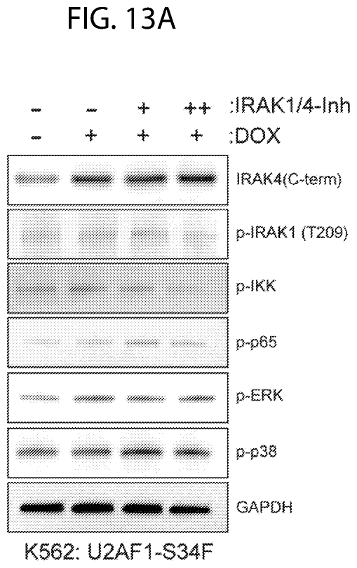

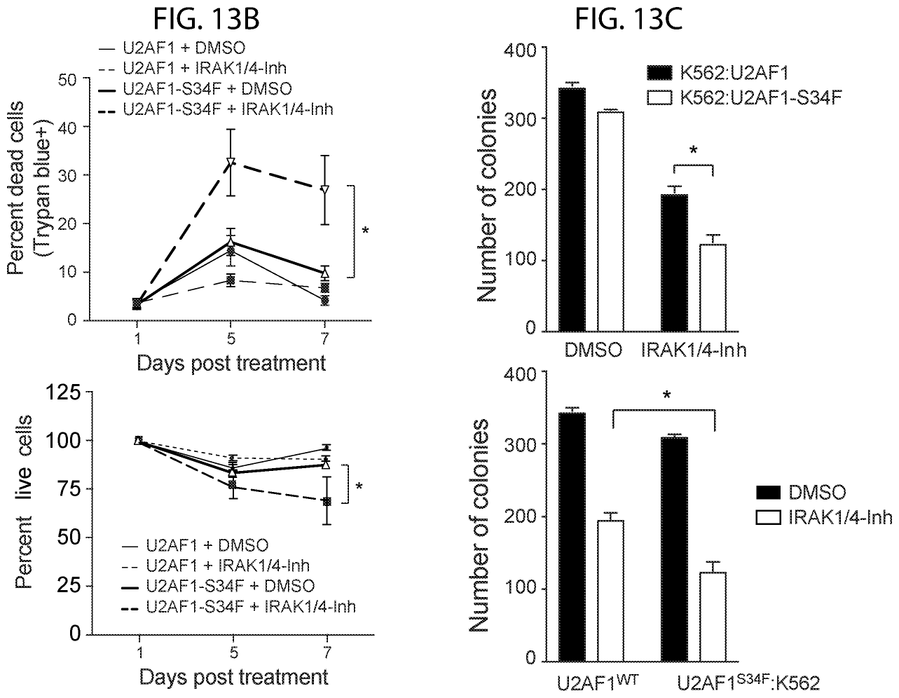

It was then investigated in whether or not there was a therapeutic opportunity in a subset of patients with increased IRAK4-Long expression, since expression of IRAK4-Long is associated with poor AML patient outcome (as described in Example 4, above). U2AF1-mutant AML cells were found to be sensitive to IRAK1/4 inhibitors, as shown in FIG. 13. Upon inhibition of IRAK1/4 by treatment with Amgen inhibitor, phospho-IRAK1, phospho-pIKK, and phospho-p65 levels were returned to normal in S34F-U2AF1 cells. K562 WT- and S34F-U2AF1 were treated with DMSO or 10 uM IRAK1/4 inhibitor for 7 days, and cell viability was monitored by trypan blue exclusion. Inhibitor treated S34F-U2AF1 cells had significantly reduced survival over 7 days. This decrease in proliferation and viability was not seen in WT-U2AF1 cells when treated with the inhibitor. WT- and S34F-U2AF1 cells were treated with DMSO or 10 uM IRAK1/4 inhibitor and plated in methylcellulose. S34F-U2AF1 cells produced significantly fewer colonies after 7 days compared to WT-U2AF1 cells. Taken together, this indicates that U2AF1 mutant cells are more sensitive to IRAK inhibition. Subsequent work has demonstrated the effectiveness of using a potent, clinical-grade compound that inhibits IRAK1/4 activity at targeting AML cells expressing IRAK4-Long, and treating patient-derived MDS and AML bone marrow cells both in vitro and in vivo (data not shown).

FIG. 13A shows results from treating K562-U2AF1-S34F cells with an IRAK1/4 inhibitor for 1 hour (+) and 2 hours (++). FIG. 13B shows results from treating K562 cells expressing wild-type or mutant U2AF1 with DMSO or IRAK1/4 inhibitor over 7 days. Viability was measured by Trypan Blue staining. FIG. 13C shows results from evaluating K562 cells expressing wild-type or mutant U2AF1 for leukemic progenitor function in methylcellulose after treatment with DMSO or IRAK1/4 inhibitor for 48 hours. Colonies were counted after 7 days. These results demonstrate that AML/MDS cells expressing the U2AF1-S34F mutation can be treated with an IRAK1/4 inhibitor in order to treat the underlying condition.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims. The claims therefore may contain different combinations of elements described in the specification and examples.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the invention. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

Bar, M., Stirewalt, D., Pogosova-Agadjanyan, E., Wagner, V., Gooley, T., Abbasi, N., Bhatia, R., Deeg, H. J., and Radich, J. (2008). Gene expression patterns in myelodyplasia underline the role of apoptosis and differentiation in disease initiation and progression. Translational oncogenomics 3, 137-149.
Boldin, M. P., Taganov, K. D., Rao, D. S., Yang, L., Zhao, J. L., Kalwani, M., Garcia-Flores, Y., Luong, M., Devrekanli, A., Xu, J., et al. (2011). miR-146a is a significant brake on autoimmunity, myeloproliferation, and cancer in mice. J Exp Med 208, 1189-1201.
Chen, G., Zeng, W., Miyazato, A., Billings, E., Maciejewski, J. P., Kajigaya, S., Sloand, E. M., and Young, N. S. (2004). Distinctive gene expression profiles of CD34 cells from patients with myelodysplastic syndrome characterized by specific chromosomal abnormalities. Blood 104, 4210-4218.
Corey, S. J., Minden, M. D., Barber, D. L., Kantarjian, H., Wang, J. C., and Schimmer, A. D. (2007). Myelodysplastic syndromes: the complexity of stem-cell diseases. Nat Rev Cancer 7,118-129.
Gondek, L. P., Tiu, R., O'Keefe, C. L., Sekeres, M. A., Theil, K. S., and Maciejewski, J. P. (2008). Chromosomal lesions and uniparental disomy detected by SNP arrays in MDS, MDS/MPD, and MDS-derived AML. Blood 111, 1534-1542.
Greenberg, P., Cox, C., LeBeau, M. M., Fenaux, P., Morel, P., Sanz, G., Sanz, M., Vallespi, T., Hamblin, T., Oscier, D., et al. (1997). International scoring system for evaluating prognosis in myelodysplastic syndromes. Blood 89, 2079-2088.
Greenberg, P. L. (2010). Current therapeutic approaches for patients with myelodysplastic syndromes. Br J Haematol 150, 131-143.
Hofmann, W. K., de Vos, S., Komor, M., Hoelzer, D., Wachsman, W., and Koeffler, H. P. (2002). Characterization of gene expression of CD34+ cells from normal and myelodysplastic bone marrow. Blood 100, 3553-3560.
Nimer, S. D. (2008). Myelodysplastic syndromes. Blood 111, 4841-4851.
Sekeres, M. A. (2010a). Are we nearer to curing patients with MDS? Best Pract Res Clin Haematol 23, 481-487.
Sokol, L., Caceres, G., Volinia, S., Alder, H., Nuovo, G. J., Liu, C. G., McGraw, K., Clark, J. A.,
Starczynowski, D. T., and Karsan, A. (2010). Innate immune signaling in the myelodysplastic syndromes. Hematol Oncol Clin North Am 24, 343-359.
Starczynowski, D. T., Kuchenbauer, F., Argiropoulos, B., Sung, S., Morin, R., Muranyi, A., Hirst, M., Hogge, D., Marra, M., Wells, R. A., et al. (2010). Identification of miR-145 and miR146a as mediators of the 5q-syndrome phenotype. Nat Med 16, 49-58.
Starczynowski, D. T., Kuchenbauer, F., Wegrzyn, J., Rouhi, A., Petriv, O., Hansen, C. L., Humphries, R. K., and Karsan, A. (2011a). MicroRNA-146a disrupts hematopoietic differentiation and survival. Exp Hematol 39, 167-178 e164.
Starczynowski, D. T., Morin, R., McPherson, A., Lam, J., Chari, R., Wegrzyn, J., Kuchenbauer, F., Hirst, M., Tohyama, K., Humphries, R. K., et al. (2011b). Genome-wide identification of human microRNAs located in leukemia-associated genomic alterations. Blood 117, 595-607.
Starczynowski, D. T., Lockwood, W. W., Delehouzee, S., Chari, R., Wegrzyn, J., Fuller, M., Tsao, M. S., Lam, S., Gazdar, A. F., Lam, W. L., and Karsan, A. (2011). TRAF6 is an amplified oncogene bridging the RAS and NF kappaB pathways in human lung cancer. J Clin Invest 121, 4095-4105. Tse, C., Shoemaker, A. R., Adickes, J., Anderson, M. G., Chen, J., Jin, S., Johnson, E. F., Marsh, K. C., Mitten, M. J., Nimmer, P., et al. (2008). ABT-263: a potent and orally bioavailable Bcl-2 family inhibitor. Cancer Res 68, 3421-3428.
Tehranchi, R., Woll, P. S., Anderson, K., Buza-Vidas, N., Mizukami, T., Mead, A. J., Astrand-Grundstrom, I., Strombeck, B., Horvat, A., Ferry, H., et al. (2010). Persistent malignant stem cells in del(5q) myelodysplasia in remission. N Engl J Med 363, 1025-1037.
Vasikova, A., Belickova, M., Budinska, E., and Cermak, J. (2010). A distinct expression of various gene subsets in CD34+ cells from patients with early and advanced myelodysplastic syndrome. Leuk Res 34, 1566-1572.
Zhao, J. L., Rao, D. S., Boldin, M. P., Taganov, K. D., O'Connell, R. M., and Baltimore, D. (2011). NF kappaB dysregulation in microRNA-146a-deficient mice drives the development of myeloid malignancies. Proc Natl Acad Sci USA 108, 9184-9189.

1. A method of treating a subject having a disease or disorder, the method comprising:
identifying a subject having a U2AF1 mutation and/or enhanced IRAK4-Long expression and/or activity relative to IRAK4-Short, as compared to a normal control; and
administering to a subject identified as having a U2AF1 mutations and/or enhanced IRAK4-Long expression and/or activity relative to IRAK4-Short, as compared to a normal control, a composition comprising an IRAK inhibitor.

2. The method of claim 1, wherein the U2AF1 mutation comprises a U2AF1-S34F mutation.

3. The method of claim 1, wherein the IRAK inhibitor inhibits IRAK4-Long activity.

4. The method of claim 1, wherein the IRAK inhibitor comprises an IRAK1/4 inhibitor.

5. The method of claim 4, wherein the IRAK1/4 inhibitor comprises an inhibitor of IRAK4-Long activity.

6. The method of claim 1, wherein the disease or disorder is associated with increased IRAK4-Long expression relative to IRAK4-Short, and increased NF-kB.

7. The method of claim 6, wherein the disease or disorder comprises myelodysplastic syndrome (MDS) or acute myeloid leukemia (AML).

8. The method of claim 7, wherein the subject has MDS comprising Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del(5q)-syndrome, chronic myelomonocytic leukemia (CMML), juvenile myelomonocytic leukemia (JMML), refractory cytopenia of childhood, or a combination thereof.

9. The method of claim 7, wherein the subject has AML comprising AML with recurrent genetic abnormalities (AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3), AML (megakaryoblastic) with a translocation between chromosomes 1 and 22, AML with myelodysplasia-related changes, AML related to previous chemotherapy or radiation (alkylating agent-related AML, topoisomerase II inhibitor-related AML), AML not otherwise categorized (AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis), myeloid sarcoma (also known as granulocytic sarcoma, chloroma or extramedullary myeloblastoma), undifferentiated and biphenotypic acute leukemias (also known as mixed phenotype acute leukemias), or a combination thereof.

10. The method of claim 7, wherein administration of an IRAK inhibitor to a subject having the U2AF1 mutation decreases the incidence of one or more symptoms associated with MDS or AML, or decreases one or more markers of viability of MDS or AML, cells.

11. The method of claim 10, wherein the one or more symptoms associated with MDS or AML comprises decreasing marrow failure, immune dysfunction, transformation to overt leukemia, or a combination thereof in the subject, or wherein the marker of viability of MDS or AML cells comprises survival over time, proliferation, growth, migration, formation of colonies, chromatic assembly, DNA binding, RNA metabolism, cell migration, cell adhesion, inflammation, or a combination thereof.

12. The method of claim 6, wherein the disease or disorder is a type of cancer comprising breast cancer, cervical cancer, colorectal cancer, endometrial cancer, glioma, head and neck cancer, liver cancer, melanoma, ovarian cancer, pancreatic cancer, prostate cancer, renal cancer, stomach cancer, testicular cancer, thyroid cancer, or urothelial cancer.

13. The method of claim 1, further comprising administration of an agent selected from an apoptotic agent, an immune modulating agent, an epigenetic modifying agent, or a combination thereof.

14. A method of assigning a subject having a disease or disorder to a specific treatment cohort, the method comprising:
determining, using a test sample from a subject having or suspected of having a disease or disorder, a presence or absence of a U2AF1 mutation and/or enhanced IRAK4-Long expression and/or activity relative to IRAK4-Short, as compared to a normal control;
assigning, where the U2AF1 mutation and/or enhanced IRAK4-Long is present, the subject to a first treatment cohort wherein the first treatment cohort is treatable by administration of an IRAK inhibitor; and
providing the cohort assignment information to a treatment facility.

15. The method of claim 14, comprising assigning, where the U2AF1 mutation and/or enhanced IRAK4-Long is absent, the subject to a second treatment cohort, wherein the second treatment cohort is not treatable, or less effectively treatable by administration of the IRAK inhibitor.

16. The method of claim 14, further comprising administering the IRAK inhibitor to the subject if the subject is in the first treatment cohort.

17. The method of claim 14, wherein the U2AF1 mutation comprises a U2AF1-S34F mutation.

18. The method of claim 14, wherein the IRAK inhibitor inhibits IRAK4-Long activity.

19. The method of claim 14, wherein the IRAK inhibitor comprises an IRAK1/4 inhibitor.

20. The method of claim 19, wherein the IRAK1/4 inhibitor comprises an inhibitor of IRAK4-Long activity.

21. The method of claim 14, further comprising administration of an IRAK1/4 inhibitor to the first treatment cohort.

22. The method of claim 14, wherein the disease or disorder is associated with increased IRAK4-Long expression relative to IRAK4-Short, and increased NF-kB.

23. The method of claim 22, wherein the disease or disorder comprises myelodysplastic syndrome (MDS) or acute myeloid leukemia (AML).

24. The method of claim 23, wherein the subject has MDS comprising Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del(5q)-syndrome, chronic myelomonocytic leukemia (CMML), juvenile myelomonocytic leukemia (JMML), refractory cytopenia of childhood, or a combination thereof.

25. The method of claim 23, wherein the subject has AML comprising AML with recurrent genetic abnormalities (AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3), AML (megakaryoblastic) with a translocation between chromosomes 1 and 22, ANIL with myelodysplasia-related changes, ANIL related to previous chemotherapy or radiation (alkylating agent-related AML, topoisomerase II inhibitor-related AML), AML not otherwise categorized (AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis), myeloid sarcoma (also known as granulocytic sarcoma, chloroma or extramedullary myeloblastoma), undifferentiated and biphenotypic acute leukemias (also known as mixed phenotype acute leukemias), or a combination thereof.

26. The method of claim 23, wherein only a fraction of AML or MDS subjects have a U2AF1 mutation that enhances IRAK4-Long expression relative to IRAK4-Short expression.

27. The method of claim 26, wherein the fraction of AML or MDS subjects having a U2AF1 mutation that enhances IRAK4-Long expression relative to IRAK4-Short expression is selected from the group consisting of less than 50%, less than 25%, less than 20%, less than 15%, less than 14%, less than 13%, less than 12%, less than 11%, less than 10%, less than 9%, less than 8%, less than 7%, less than 5%, less than 4%, less than 3%, less than 2%, or less than 1%.

28. The method of claim 23, wherein administration of an IRAK inhibitor to a subject having the U2AF1 mutation decreases the incidence of one or more symptoms associated with MDS or AML or decreases one or more markers of viability of MDS or AML cells.

29. The method of claim 28, wherein the one or more symptoms associated with MDS or AML comprises decreasing marrow failure, immune dysfunction, transformation to overt leukemia, or a combination thereof in the subject, or wherein the marker of viability of MDS or AML cells comprises survival over time, proliferation, growth, migration, formation of colonies, chromatic assembly, DNA binding, RNA metabolism, cell migration, cell adhesion, inflammation, or a combination thereof.

30. The method of claim 22, wherein the disease or disorder is a type of cancer comprising breast cancer, cervical cancer, colorectal cancer, endometrial cancer, glioma, head and neck cancer, liver cancer, melanoma, ovarian cancer, pancreatic cancer, prostate cancer, renal cancer, stomach cancer, testicular cancer, thyroid cancer, or urothelial cancer.

31. The method of claim 14, further comprising administration of an agent selected from an apoptotic agent, an immune modulating agent, an epigenetic modifying agent, or a combination thereof.

32. The method of claim 14, wherein the subject in the first treatment cohort or the second treatment cohort is enrolled in a clinical trial.

33. A method for improving a clinical trial for treating a disease or disorder, the method comprising:
determining, using a test sample from one or more subjects having or suspected of having a disease or disorder, a presence or absence of a U2AF1 mutation and/or enhanced IRAK4-Long expression and/or activity relative to IRAK4-Short, as compared to a normal control;
assigning, where the U2AF1 mutation and/or enhanced IRAK4-Long is present, the subject to a first treatment cohort, assigning, where the U2AF1 mutation and/or enhanced IRAK4-Long is absent, the sample to a second treatment cohort; and
administering an IRAK inhibitor to the subject only if the subject is in the first treatment cohort.

34. The method of claim 33, wherein the first treatment cohort is treatable by administration of an IRAK inhibitor, and wherein the second treatment cohort is not treatable, or less effectively treatable by administration of the IRAK inhibitor.

35. The method of claim 33, wherein the U2AF1 mutation comprises a U2AF1-S34F mutation.

36. The method of claim 33, wherein the IRAK inhibitor inhibits IRAK4-Long activity.

37. The method of claim 33, wherein the IRAK inhibitor comprises an IRAK1/4 inhibitor.

38. The method of claim 37, wherein the IRAK1/4 inhibitor comprises an inhibitor of IRAK4-Long activity.

39. The method of claim 33, further comprising administration of an IRAK1/4 inhibitor to the first treatment cohort.

40. The method of claim 33, wherein the disease or disorder is associated with increased IRAK4-Long expression relative to IRAK4-Short, and increased NF-kB.

41. The method of claim 40, wherein the disease or disorder comprises myelodysplastic syndrome (MDS) or acute myeloid leukemia (AML).

42. The method of claim 41, wherein the subject has MDS comprising Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del(5q)-syndrome, chronic myelomonocytic leukemia (CMML), juvenile myelomonocytic leukemia (JMML), refractory cytopenia of childhood, or a combination thereof.

43. The method of claim 41, wherein the subject has AML comprising AML with recurrent genetic abnormalities (AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3), AML (megakaryoblastic) with a translocation between chromosomes 1 and 22, AML with myelodysplasia-related changes, AML related to previous chemotherapy or radiation (alkylating agent-related AML, topoisomerase II inhibitor-related AML), AML not otherwise categorized (AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis), myeloid sarcoma (also known as granulocytic sarcoma, chloroma or extramedullary myeloblastoma), undifferentiated and biphenotypic acute leukemias (also known as mixed phenotype acute leukemias), or a combination thereof.

44. The method of claim 41, wherein only a fraction of AML or MDS subjects have a U2AF1 mutation that enhances IRAK4-Long expression relative to IRAK4-Short expression.

45. The method of claim 44, wherein the fraction of AML or MDS subjects having a U2AF1 mutation that enhances IRAK4-Long expression relative to IRAK4-Short expression is selected from the group consisting of less than 50%, less than 25%, less than 20%, less than 15%, less than 14%, less than 13%, less than 12%, less than 11%, less than 10%, less than 9%, less than 8%, less than 7%, less than 5%, less than 4%, less than 3%, less than 2%, or less than 1%.

46. The method of claim 41, wherein administration of an IRAK inhibitor to a subject having the U2AF1 mutation decreases the incidence of one or more symptoms associated with MDS or AML or decreases one or more markers of viability of MDS or AML cells.

47. The method of claim 46, wherein the one or more symptoms associated with MDS or AML comprises decreasing marrow failure, immune dysfunction, transformation to overt leukemia, or a combination thereof in the subject, or wherein the marker of viability of MDS or AML cells comprises survival over time, proliferation, growth, migration, formation of colonies, chromatic assembly, DNA binding, RNA metabolism, cell migration, cell adhesion, inflammation, or a combination thereof.

48. The method of claim 40, wherein the disease or disorder is a type of cancer comprising breast cancer, cervical cancer, colorectal cancer, endometrial cancer, glioma, head and neck cancer, liver cancer, melanoma, ovarian cancer, pancreatic cancer, prostate cancer, renal cancer, stomach cancer, testicular cancer, thyroid cancer, or urothelial cancer.

49. The method of claim 33, further comprising administration of an agent selected from an apoptotic agent, an immune modulating agent, an epigenetic modifying agent, or a combination thereof.
